SEQUENCE LISTING (73 sequences)

SEQ ID NO: 1 - skipped

SEQ ID NO: 2 - DNA, 10 bp, synthetic construct
  misc_feature 1..10: note = G QUADRAPLEX
  source 1..10: mol_type = other DNA
gccgaaaggc

SEQ ID NO: 3 - DNA, 16 bp, synthetic construct
  misc_feature 1..16: note = G QUADRAPLEX
  source 1..16: mol_type = other DNA
gataccgaaaggtatc

SEQ ID NO: 4 - DNA, 22 bp, synthetic construct
  misc_feature 1..22: note = G QUADRAPLEX
  source 1..22: mol_type = other DNA
gatctgaccgaaaggtcagatc

SEQ ID NO: 5 - DNA, 28 bp, synthetic construct
  misc_feature 1..28: note = G QUAD
  source 1..28: mol_type = other DNA
gatcgtctgaccgaaaggtcagacgatc

SEQ ID NO: 6 - DNA, 34 bp, synthetic construct
  misc_feature 1..34: note = G QUAD
  source 1..34: mol_type = other DNA
gaggctcgtctgaccgaaaggtcagacgagcctc

SEQ ID NO: 7 - DNA, 44 bp, synthetic construct
  misc_feature 1..44: note = G QUAD
  source 1..44: mol_type = other DNA
gaggcttctagcgtctgaccgaaaggtcagacgctagaagcctc

SEQ ID NO: 8 - DNA, 11 bp, synthetic construct
  misc_feature 1..11: note = G QUAD
  source 1..11: mol_type = other DNA
ggtggtggtgg

SEQ ID NO: 9 - DNA, 20 bp, synthetic construct
  misc_feature 1..20: note = G QUAD
  source 1..20: mol_type = other DNA
ggaggaggaggaggaggagg

SEQ ID NO: 10 - DNA, 20 bp, synthetic construct
  misc_feature 1..20: note = G QUAD
  source 1..20: mol_type = other DNA
ggaggtggaggtggaggtgg

SEQ ID NO: 11 - DNA, 23 bp, synthetic construct
  misc_feature 1..23: note = G QUAD
  source 1..23: mol_type = other DNA
ggcctgtggcctgtggcctgtgg

SEQ ID NO: 12 - DNA, 26 bp, synthetic construct
  misc_feature 1..26: note = G QUAD
  source 1..26: mol_type = other DNA
ggcctgttggcctgttggcctgttgg

SEQ ID NO: 13 - DNA, 29 bp, synthetic construct
  misc_feature 1..29: note = G QUAD
  source 1..29: mol_type = other DNA
ggtagcattggtagcattggtagcattgg

SEQ ID NO: 14 - DNA, 11 bp, synthetic construct
  misc_feature 1..11: note = G QUAD
  source 1..11: mol_type = other DNA
ggaggaggagg

SEQ ID NO: 15 - DNA, 11 bp, synthetic construct
  misc_feature 1..11: note = G QUAD
  source 1..11: mol_type = other DNA
ggcggcggcgg

SEQ ID NO: 16 - DNA, 14 bp, synthetic construct
  misc_feature 1..14: note = G QUAD
  source 1..14: mol_type = other DNA
ggaaggaaggaagg

SEQ ID NO: 17 - DNA, 14 bp, synthetic construct
  misc_feature 1..14: note = G QUAD
  source 1..14: mol_type = other DNA
ggctggctggctgg

SEQ ID NO: 18 - DNA, 14 bp, synthetic construct
  misc_feature 1..14: note = G QUAD
  source 1..14: mol_type = other DNA
ggcaggcaggcagg

SEQ ID NO: 19 - DNA, 17 bp, synthetic construct
  misc_feature 1..17: note = G QUAD
  source 1..17: mol_type = other DNA
ggagaggagaggagagg

SEQ ID NO: 20 - DNA, 17 bp, synthetic construct
  misc_feature 1..17: note = G QUAD
  source 1..17: mol_type = other DNA
ggaaaggaaaggaaagg

SEQ ID NO: 21 - DNA, 17 bp, synthetic construct
  misc_feature 1..17: note = G QUAD
  source 1..17: mol_type = other DNA
ggtcaggtcaggtcagg

SEQ ID NO: 22 - DNA, 15 bp, synthetic construct
  misc_feature 1..15: note = G QUAD
  source 1..15: mol_type = other DNA
gggtgggtgggtggg

SEQ ID NO: 23 - DNA, 24 bp, synthetic construct
  misc_feature 1..24: note = G QUAD
  source 1..24: mol_type = other DNA
gggaggagggaggagggaggaggg

SEQ ID NO: 24 - DNA, 24 bp, synthetic construct
  misc_feature 1..24: note = G QUAD
  source 1..24: mol_type = other DNA
gggaggtgggaggtgggaggtggg

SEQ ID NO: 25 - DNA, 27 bp, synthetic construct
  misc_feature 1..27: note = G QUAD
  source 1..27: mol_type = other DNA
gggcctgtgggcctgtgggcctgtggg

SEQ ID NO: 26 - DNA, 30 bp, synthetic construct
  misc_feature 1..30: note = G QUAD
  source 1..30: mol_type = other DNA
gggcctgttgggcctgttgggcctgttggg

SEQ ID NO: 27 - DNA, 33 bp, synthetic construct
  misc_feature 1..33: note = G QUAD
  source 1..33: mol_type = other DNA
gggtagcattgggtagcattgggtagcattggg

SEQ ID NO: 28 - DNA, 15 bp, synthetic construct
  misc_feature 1..15: note = G QUAD
  source 1..15: mol_type = other DNA
gggagggagggaggg

SEQ ID NO: 29 - DNA, 15 bp, synthetic construct
  misc_feature 1..15: note = G QUAD
  source 1..15: mol_type = other DNA
gggcgggcgggcggg

SEQ ID NO: 30 - DNA, 18 bp, synthetic construct
  misc_feature 1..18: note = G QUAD
  source 1..18: mol_type = other DNA
gggaagggaagggaaggg

SEQ ID NO: 31 - DNA, 18 bp, synthetic construct
  misc_feature 1..18: note = G QUAD
  source 1..18: mol_type = other DNA
gggctgggctgggctggg

SEQ ID NO: 32 - DNA, 18 bp, synthetic construct
  misc_feature 1..18: note = G QUAD
  source 1..18: mol_type = other DNA
gggcagggcagggcaggg

SEQ ID NO: 33 - DNA, 21 bp, synthetic construct
  misc_feature 1..21: note = G QUAD
  source 1..21: mol_type = other DNA
gggagagggagagggagaggg

SEQ ID NO: 34 - DNA, 21 bp, synthetic construct
  misc_feature 1..21: note = G QUAD
  source 1..21: mol_type = other DNA
gggaaagggaaagggaaaggg

SEQ ID NO: 35 - DNA, 21 bp, synthetic construct
  misc_feature 1..21: note = G QUAD
  source 1..21: mol_type = other DNA
gggtcagggtcagggtcaggg

SEQ ID NO: 36 - DNA, 19 bp, synthetic construct
  misc_feature 1..19: note = G QUAD
  source 1..19: mol_type = other DNA
ggggtggggtggggtgggg

SEQ ID NO: 37 - DNA, 28 bp, synthetic construct
  misc_feature 1..28: note = G QUAD
  source 1..28: mol_type = other DNA
ggggaggaggggaggaggggaggagggg

SEQ ID NO: 38 - DNA, 28 bp, synthetic construct
  misc_feature 1..28: note = G QUAD
  source 1..28: mol_type = other DNA
ggggaggtggggaggtggggaggtgggg

SEQ ID NO: 39 - DNA, 31 bp, synthetic construct
  misc_feature 1..31: note = G QUAD
  source 1..31: mol_type = other DNA
ggggcctgtggggcctgtggggcctgtgggg

SEQ ID NO: 40 - DNA, 34 bp, synthetic construct
  misc_feature 1..34: note = G QUAD
  source 1..34: mol_type = other DNA
ggggcctgttggggcctgttggggcctgttgggg

SEQ ID NO: 41 - DNA, 37 bp, synthetic construct
  misc_feature 1..37: note = G QUAD
  source 1..37: mol_type = other DNA
ggggtagcattggggtagcattggggtagcattgggg

SEQ ID NO: 42 - DNA, 19 bp, synthetic construct
  misc_feature 1..19: note = G QUAD
  source 1..19: mol_type = other DNA
ggggaggggaggggagggg

SEQ ID NO: 43 - DNA, 19 bp, synthetic construct
  misc_feature 1..19: note = G QUAD
  source 1..19: mol_type = other DNA
ggggcggggcggggcgggg

SEQ ID NO: 44 - DNA, 22 bp, synthetic construct
  misc_feature 1..22: note = G QUAD
  source 1..22: mol_type = other DNA
ggggaaggggaaggggaagggg

SEQ ID NO: 45 - DNA, 22 bp, synthetic construct
  misc_feature 1..22: note = G QUAD
  source 1..22: mol_type = other DNA
ggggctggggctggggctgggg

SEQ ID NO: 46 - DNA, 22 bp, synthetic construct
  misc_feature 1..22: note = G QUAD
  source 1..22: mol_type = other DNA
ggggcaggggcaggggcagggg

SEQ ID NO: 47 - DNA, 25 bp, synthetic construct
  misc_feature 1..25: note = G QUAD
  source 1..25: mol_type = other DNA
ggggagaggggagaggggagagggg

SEQ ID NO: 48 - DNA, 25 bp, synthetic construct
  misc_feature 1..25: note = G QUAD
  source 1..25: mol_type = other DNA
ggggaaaggggaaaggggaaagggg

SEQ ID NO: 49 - DNA, 25 bp, synthetic construct
  misc_feature 1..25: note = G QUAD
  source 1..25: mol_type = other DNA
ggggtcaggggtcaggggtcagggg

SEQ ID NO: 50 - DNA, 10 bp, synthetic construct
  misc_feature 1..10: note = HAIRPIN
  source 1..10: mol_type = other DNA
gccgtgaggc

SEQ ID NO: 51 - DNA, 16 bp, synthetic construct
  misc_feature 1..16: note = HAIRPIN
  source 1..16: mol_type = other DNA
gataccgtgaggtatc

SEQ ID NO: 52 - DNA, 22 bp, synthetic construct
  misc_feature 1..22: note = HAIRPIN
  source 1..22: mol_type = other DNA
gatctgaccgtgaggtcagatc

SEQ ID NO: 53 - DNA, 28 bp, synthetic construct
  misc_feature 1..28: note = HAIRPIN
  source 1..28: mol_type = other DNA
gatcgtctgaccgtgaggtcagacgatc

SEQ ID NO: 54 - DNA, 34 bp, synthetic construct
  misc_feature 1..34: note = HAIRPIN
  source 1..34: mol_type = other DNA
gaggctcgtctgaccgtgaggtcagacgagcctc

SEQ ID NO: 55 - DNA, 44 bp, synthetic construct
  misc_feature 1..44: note = HAIRPIN
  source 1..44: mol_type = other DNA
gaggcttctagcgtctgaccgtgaggtcagacgctagaagcctc

SEQ ID NO: 56 - DNA, 18 bp, synthetic construct
  misc_feature 1..18: note = KNOT
  source 1..18: mol_type = other DNA
gcgacttcggtcgccgaa

SEQ ID NO: 57 - DNA, 24 bp, synthetic construct
  misc_feature 1..24: note = KNOT
  source 1..24: mol_type = other DNA
gcgacttcgcatgtcgcatgcgaa

SEQ ID NO: 58 - DNA, 32 bp, synthetic construct
  misc_feature 1..32: note = KNOT
  source 1..32: mol_type = other DNA
gcgacttcgcatagacgtcgcgtctatgcgaa

SEQ ID NO: 59 - DNA, 24 bp, synthetic construct
  misc_feature 1..24: note = KNOT
  source 1..24: mol_type = other DNA
gcgactagttcgctagtcgccgaa

SEQ ID NO: 60 - DNA, 30 bp, synthetic construct
  misc_feature 1..30: note = KNOT
  source 1..30: mol_type = other DNA
gcgactagttcgcatctagtcgcatgcgaa

SEQ ID NO: 61 - DNA, 38 bp, synthetic construct
  misc_feature 1..38: note = KNOT
  source 1..38: mol_type = other DNA
gcgactagttcgcatagacctagtcgcgtctatgcgaa

SEQ ID NO: 62 - DNA, 32 bp, synthetic construct
  misc_feature 1..32: note = KNOT
  source 1..32: mol_type = other DNA
gagctagcatcattcgtgatgctagctccgaa

SEQ ID NO: 63 - DNA, 38 bp, synthetic construct
  misc_feature 1..38: note = KNOT
  source 1..38: mol_type = other DNA
gagctagcatcattcgcattgatgctagctcatgcgaa

SEQ ID NO: 64 - DNA, 46 bp, synthetic construct
  misc_feature 1..46: note = KNOT
  source 1..46: mol_type = other DNA
gagctagcatcattcgcatagactgatgctagctcgtctatgcgaa

SEQ ID NO: 65 - DNA, 10 bp, synthetic construct
  misc_feature 1..10: note = HAIRPIN
  source 1..10: mol_type = other DNA
gccttcgggc

SEQ ID NO: 66 - DNA, 16 bp, synthetic construct
  misc_feature 1..16: note = HAIRPIN
  source 1..16: mol_type = other DNA
gataccttcgggtatc

SEQ ID NO: 67 - DNA, 22 bp, synthetic construct
  misc_feature 1..22: note = HAIRPIN
  source 1..22: mol_type = other DNA
gatctgaccttcgggtcagatc

SEQ ID NO: 68 - DNA, 28 bp, synthetic construct
  misc_feature 1..28: note = HAIRPIN
  source 1..28: mol_type = other DNA
gatcgtctgaccttcgggtcagacgatc

SEQ ID NO: 69 - DNA, 34 bp, synthetic construct
  misc_feature 1..34: note = HAIRPIN
  source 1..34: mol_type = other DNA
gaggctcgtctgaccttcgggtcagacgagcctc

SEQ ID NO: 70 - DNA, 44 bp, synthetic construct
  misc_feature 1..44: note = HAIRPIN
  source 1..44: mol_type = other DNA
gaggcttctagcgtctgaccttcgggtcagacgctagaagcctc

SEQ ID NO: 71 - DNA, 50 bp, synthetic construct
  misc_feature 1..50: note = EXO
  source 1..50: mol_type = other DNA
ggcaaggtacggattagccgtaggggcttgagaaccccccctccccactc

SEQ ID NO: 72 - DNA, 93 bp, synthetic construct
  misc_feature 1..93: note = EXO
  source 1..93: mol_type = other DNA
cacagatcatggaatgatgcggcagcgcgcgagagcgacggggaagtggtcgcacccgacgcaccatccatgaagcaatacttcgtgagaccc

SEQ ID NO: 73 - DNA, 57 bp, synthetic construct
  misc_feature 1..57: note = EXO
  source 1..57: mol_type = other DNA
gggtcaggccggcgaaagtcgccacagtttggggaaagctgtgcagcctgtaacccc

(19) United States
(12) Patent Application Publication
(10) Pub. No.: US 2024/0018580 A1
(43) Pub. Date: Jan. 18, 2024

(54) gRNA STABILIZATION IN NUCLEIC ACID-GUIDED NICKASE EDITING

(71) Applicant: Inscripta, Inc., Pleasanton, CA (US)

(72) Inventor: Aamir Mir, Pleasanton, CA (US)

(73) Assignee: Inscripta, Inc., Pleasanton, CA (US)

(21) Appl. No.: 18/460,058

(22) Filed: Sep. 1, 2023

Related U.S. Application Data
(63) Continuation of application No. 17/538,166, filed on Nov. 30, 2021.
(60) Provisional application No. 63/122,339, filed on Dec. 7, 2020.

(51) Int. Cl. C12Q 1/6865

(52) U.S. Cl. CPC C12Q 1/6865

(57) ABSTRACT

The present disclosure provides compositions of matter, methods and instruments for nucleic acid-guided nickase/reverse transcriptase fusion editing in live cells. Editing efficiency is improved using fusion proteins (e.g., the nickase-RT fusion) that retain certain characteristics of nucleic acid-directed nucleases (e.g., the binding specificity and ability to cleave one or more DNA strands in a targeted manner) combined with reverse transcriptase activity. Editing cassettes are employed, comprising a gRNA and a repair template where the 3′ end of the repair template is protected from degradation.

### RELATED CASES

This application claims priority to U.S. Ser. No. 63/122,339 filed 7 Dec. 2020, entitled “gRNA STABILIZATION IN NUCLEIC ACID-GUIDED NICKASE EDITING” which is incorporated by reference in its entirety.

### FIELD OF THE INVENTION

This invention relates to compositions of matter, methods and instruments for improved nucleic acid-guided nickase editing of live cells, particularly mammalian cells.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the methods referenced herein do not constitute prior art under the applicable statutory provisions.

The ability to make precise, targeted changes to the genome of living cells has been a long-standing goal in biomedical research and development. Recently various nucleases have been identified that allow manipulation of gene sequence, and hence gene function. The nucleases include nucleic acid guided nucleases, which enable researchers to generate permanent edits in live cells. Of course, it is desirable to attain the highest editing rates possible in a cell population; however, in many instances the percentage of edited cells resulting from nucleic acid-guided nuclease editing can be in the single digits.

There is thus a need in the art of nucleic acid-guided nuclease editing for improved methods, compositions, modules and instruments for increasing the efficiency of editing. The present disclosure addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present disclosure relates to methods and compositions for stabilizing gRNAs during nucleic acid-guided nickase editing. With the present compositions and methods, editing efficiency is improved using nucleic acid-guided nickase/reverse transcriptase fusion proteins (e.g., nickase-RT fusion proteins) that retain certain characteristics of nucleic acid-directed nucleases (e.g., the binding specificity and ability to cleave one or more DNA strands in a targeted manner) combined with another enzymatic activity such as reverse transcriptase activity. The nickase-RT fusion enzyme is used with a CF editing cassette (“CREATE fusion editing cassette”) comprising a gRNA and repair template where the CF editing cassette is protected at the 3′ end of the repair template with an RNA stabilization moiety.

Thus, there is provided a CREATE fusion editing cassette for performing nucleic acid-guided nickase/reverse transcriptase fusion editing comprising from 3′ to 5′: 1) an RNA repair template comprising: an RNA stabilization moiety; a linker region; a primer binding region capable of binding to a nicked target DNA; a nick-to-edit region; and a region of post-edit homology; and 2) a gRNA comprising: a guide sequence; and a scaffold region.

In some aspects, the RNA stabilization moiety is a G quadraplex, an RNA hairpin, an RNA pseudoknot or an exoribonuclease resistant RNA. In some aspects, the RNA stabilization moiety is a G quadraplex, and in some aspects, the G quadraplex is selected from SEQ ID No: 1; SEQ ID No: 2; SEQ ID No: 3: SEQ ID No: 4; SEQ ID No: 5; SEQ ID No: 6; SEQ ID No: 7; SEQ ID No: 8; SEQ ID No: 9; SEQ ID No: 10; SEQ ID No: 11; SEQ ID No: 12; SEQ ID No: 13; SEQ ID No: 14; SEQ ID No: 15: SEQ ID No: 16; SEQ ID No: 17; SEQ ID No: 18: SEQ ID No: 19; SEQ ID No: 20; SEQ ID No: 21; SEQ ID No: 22; SEQ ID No: 23; SEQ ID No: 24; SEQ ID No: 25; SEQ ID No: 26; SEQ ID No: 27; SEQ ID No: 28; SEQ ID No: 29; SEQ ID No: 30; SEQ ID No: 31; SEQ ID No: 32; SEQ ID No: 33; SEQ ID No: 34; SEQ ID No: 35; SEQ ID No: 36; SEQ ID No: 37; SEQ ID No: 38; SEQ ID No: 39; SEQ ID No: 40; SEQ ID No: 41: SEQ ID No: 42; SEQ ID No: 43; SEQ ID No: 44; SEQ ID No: 45; SEQ ID No: 46; SEQ ID No: 47; SEQ ID No: 48; and SEQ ID No: 49. In some aspects, the RNA stabilization moiety is an RNA hairpin; and in some aspects, the RNA hairpin selected from SEQ ID No: 50; SEQ ID No: 51; SEQ ID No: 52: SEQ ID No: 53: SEQ ID No: 54: SEQ ID No: 55; SEQ ID No: 65; SEQ ID No: 66; SEQ ID No: 67; SEQ ID No: 68; SEQ ID No: 69; and SEQ ID No: 70. In some aspects, the RNA stabilization moiety is an RNA pseudoknot where the RNA pseudoknot is selected from SEQ ID No: 50; SEQ ID No: 56; SEQ ID No: 57; SEQ ID No: 58; SEQ ID No: 59; SEQ ID No: 60; SEQ ID No: 61; SEQ ID No: 62; SEQ ID No: 63; and SEQ ID No: 64. In some aspects, the RNA stabilization moiety is an exoribonuclease resistant RNA, and in some aspects, the exoribonuclease resistant RNA is selected from SEQ ID No: 71; SEQ ID No: 72; and SEQ ID No: 73.

In some aspects, the CREATE fusion editing cassette has a linker region from 0 to 20 nucleotides in length. In some aspects, the CREATE fusion editing cassette has a primer binding region from 0 to 20 nucleotides in length. In some aspects, the CREATE fusion editing cassette has a nick-to-edit region from 0 to 20 nucleotides in length. In some aspects, the CREATE fusion editing cassette has a region of post-edit homology 3 to 20 nucleotides in length. In some aspects, the CREATE fusion editing cassette has a guide sequence capable of hybridizing to a genomic target locus and a scaffold sequence is capable of interacting or complexing with a nucleic acid-guided nuclease.

These aspects and other features and advantages of the invention are described below in more detail.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1A is a simplified block diagram of an exemplary method for editing live cells via nucleic acid-guided nickase/reverse transcriptase fusion (“nickase-RT fusion”) editing. FIG. 1B is an alternative simplified block diagram of an exemplary method for editing live cells via nickase-RT fusion editing. FIG. 1C is a simplified graphic depiction of a nucleic acid-guided nickase enzyme/reverse transcriptase fusion protein (nickase-RT fusion) and a CF editing cassette. FIG. 1D is a simplified graphic depiction of a nucleic acid-guided nickase enzyme/reverse transcriptase fusion protein (nickase-RT fusion) and a CF editing cassette comprising a gRNA and a repair template comprising an RNA stabilization moiety (here a G2 quadraplex, hairpin, pseudoknot) at the 3′ end of the repair template (i.e., a “3′ protected CF editing cassette” or “stabilized CF editing cassette” or “StCFEC”). FIG. 1E shows depictions of the generalized pseudoknot structure tested as an RNA stabilization moiety.

FIGS. 2A-2C depict three different views of an exemplary automated multi-module cell processing instrument for performing nickase-RT fusion editing.

FIGS. 3A-3C depict various views and components of exemplary embodiments of a bioreactor module included in an integrated instrument useful for growing and transfecting cells for performing nickase-RT fusion editing. FIGS. 3D and 3E depict an exemplary integrated instrument for growing and transfecting cells for performing nickase-RT fusion editing.

FIG. 4A depicts an exemplary workflow employing microcarrier-partitioned delivery for cells for performing nickase-RT fusion editing of mammalian cells grown in suspension. FIG. 4B depicts an option for growing, passaging, transfecting and editing iPSCs (induced pluripotent stem cells) involving sequential transduction and transfection of CF editing cassettes and nickase-RT fusion enzymes. FIG. 4C depicts an exemplary workflow employing microcarrier-partitioned delivery for performing nickase-RT fusion editing of mammalian cells. FIG. 4D depicts an alternative workflow employing microcarrier-partitioned delivery for performing nickase-RT fusion editing of mammalian cells.

FIG. 5 is a simplified process diagram of an embodiment of an exemplary automated multi-module cell processing instrument comprising a solid wall selection/singulation/growth/induction/editing/normalization device for recursive cell editing—including mammalian cell editing—in a system using a nickase-RT fusion enzyme and a CR editing cassette with a gRNA stabilization moiety (StCFEC) at the 3′ end of the repair template component of the CF editing cassette.

FIG. 6 comprises two graphs reporting results demonstrating that CF editing cassettes with 3′ gRNA stabilization moieties (StCFECs) increase editing in the GFP-to-BFP system.

FIG. 7 is a bar graph showing that single copy number (SCN) delivery of StCFECs increases editing over CF editing cassettes without an RNA stabilization moiety.

FIG. 8 is a simplified graphic of experimental design for determining cell viability and editing efficiency.

FIG. 9 is a bar graph showing >90% transfection efficiency of StCFEC mRNA.

FIG. 10 is a bar graph confirming single- and multiple-copy CF editing cassette integrations in various iPSC lines.

FIG. 11 is a bar graph showing cell viability at 96 hours post-transfection of nuclease mRNA (Cas9 and MAD2007 nickase-RT fusion protein) in different iPSC lines under different CF editing cassette and StCFEC lentivirus transfection dilutions.

FIG. 12 demonstrates the low indel rates observed in iPSC lines using the MAD2007 nickase-RT fusion protein.

FIG. 13 is a bar graph showing lenti-integrated CF editing cassettes comprising RNA stability moieties confer robust editing as compared to CF editing cassettes without stabilization moieties across five iPSC lines.

FIG. 14 is a graphic depicting the screening workflow to determine editing efficiency for various putative 3′ stabilization moieties.

FIGS. 15A and 15B are a bar graphs reporting the editing rate for CF editing cassettes comprising the various putative 3′ RNA stabilization moieties listed in Table 1 vis-à-vis the G2 quadraplex CF editing cassettes and the CFg5 editing cassette (unprotected).

FIG. 16A is a bar graph of GFP to BFP edit rates 120 hours post-transfection in PGP168_G2B iPSCs. FIG. 16B is a bar graph of GFP to BFP edit rates 120 hours post-transfection in WTC11_G2B iPSCs.

FIG. 17 shows the improvement in editing rates for viral exoribonuclease-resistant RNAs used as 3′ stabilization moieties in CF editing cassettes.

It should be understood that the drawings are not necessarily to scale, and that like reference numbers refer to like features.

### DETAILED DESCRIPTION

All of the functionalities described in connection with one embodiment are intended to be applicable to the additional embodiments described herein except where expressly stated or where the feature or function is incompatible with the additional embodiments. For example, where a given feature or function is expressly described in connection with one embodiment but not expressly mentioned in connection with an alternative embodiment, it should be understood that the feature or function may be deployed, utilized, or implemented in connection with the alternative embodiment unless the feature or function is incompatible with the alternative embodiment.

The practice of the techniques described herein may employ, unless otherwise indicated, conventional techniques and descriptions of organic chemistry, polymer technology, molecular biology (including recombinant techniques), cell biology, biochemistry and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent procedures can, of course, also be used. Such techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003). PCR Primer: A Laboratory Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” (1984), IRL Press, London; Nelson and Cox (2000), Lehninger, Principles of Biochemistry 3rd Ed., W. H. Freeman Pub., New York, N.Y.; Berg, et al. (2002) Biochemistry, 5th Ed., W.H. Freeman Pub., New York, N.Y.; all of which are herein incorporated in their entirety by reference for all purposes. CRISPR-specific techniques can be found in, e.g., Genome Editing and Engineering from TALENs and CRISPRs to Molecular Surgery, Appasani and Church (2018); and CRISPR: Methods and Protocols, Lindgren and Charpentier (2015); both of which are herein incorporated in their entirety by reference for all purposes.

Note that as used herein and in the appended claims, the singular forms “a,” “an,” and “the” include plural referents unless the context clearly dictates otherwise. Thus, for example, reference to “an oligonucleotide” refers to one or more oligonucleotides, and reference to “an automated system” includes reference to equivalent steps and methods for use with the system known to those skilled in the art, and so forth. Additionally, it is to be understood that terms such as “left,” “right,” “top,” “bottom,” “front,” “rear,” “side,” “height,” “length,” “width,” “upper,” “lower,” “interior,” “exterior,” “inner,” “outer” that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, methods and cell populations that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of ordinary skill in the art that the present invention may be practiced without one or more of these specific details. In other instances, well-known features and procedures well known to those skilled in the art have not been described in order to avoid obscuring the invention.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3.

The term DNA “control sequences” refers collectively to promoter sequences, polyadenylation signals, transcription termination sequences, upstream regulatory domains, origins of replication, internal ribosome entry sites, nuclear localization sequences, enhancers, and the like, which collectively provide for the replication, transcription and translation of a coding sequence in a recipient cell. Not all of these types of control sequences need to be present so long as a selected coding sequence is capable of being replicated, transcribed and-for some components-translated in an appropriate host cell.

The terms “CREATE fusion editing cassette” or “CF editing cassette” refer to a nucleic acid molecule comprising a coding sequence for transcription of a gRNA covalently linked to a coding sequence for transcription of a repair template for use with nickase-RT fusion enzymes. For additional information regarding traditional editing cassettes, e.g., comprising a gRNA and a repair template for use in nucleic acid-guided nuclease systems, see U.S. Pat. Nos. 9,982,278; 10,266,849; 10,240,167; 10,351,877; 10,364,442; 10,435,715; 10,465,207; 10,669,559; 10,771,284; 10,731,498; and 11,078,498, all of which are incorporated by reference herein.

The terms “CREATE fusion editing system” or “CF editing system” refer to the combination of a nucleic acid-guided nickase enzyme/reverse transcriptase fusion protein (“nickase-RT fusion”) and a CREATE fusion editing cassette (“CF editing cassette”) to effect editing in live cells.

The terms “guide nucleic acid” or “guide RNA” or “gRNA” refer to a polynucleotide comprising 1) a guide sequence capable of hybridizing to a genomic target locus, and 2) a scaffold sequence capable of interacting or complexing with a nucleic acid-guided nuclease.

“Homology” or “identity” or “similarity” refers to sequence similarity between two peptides or, more often in the context of the present disclosure, between two nucleic acid molecules. The term “homologous region” refers to a region on the gRNA or repair template with a certain degree of homology with the target DNA sequence. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences.

As used herein, “nucleic acid-guided nickase/reverse transcriptase fusion” or “nickase-RT fusion” or “nickase-RT fusion enzyme” refer to a nucleic acid-guided nickase or nucleic acid-guided nuclease or CRISPR nuclease that has been engineered to act as a nickase rather than a nuclease that initiates double-stranded DNA breaks, and where the nucleic acid-guided nickase is fused to a reverse transcriptase, which is an enzyme used to generate cDNA from an RNA template. Utilization of a nickase-RT fusion enzyme along with a CF editing cassette incorporates an edit in the DNA target sequence at the RNA level through reverse transcription of the repair template rather than at the DNA level such as through homologous recombination. For information regarding nickase-RT fusions see, e.g., U.S. Pat. No. 10,689,669 and U.S. Ser. No. 16/740,421.

The term “nickase-RT editing components” refers to one or both of a nickase-RT fusion enzyme and a CF editing cassette, where the CF editing cassette may comprise an RNA stabilization moiety (“StCFEC”) or no RNA stabilization moiety.

“Operably linked” refers to an arrangement of elements where the components so described are configured so as to perform their usual function. Thus, control sequences operably linked to a coding sequence are capable of effecting the transcription, and in some cases, the translation, of a coding sequence. The control sequences need not be contiguous with the coding sequence so long as they function to direct the expression of the coding sequence. Thus, for example, intervening untranslated yet transcribed sequences can be present between a promoter sequence and the coding sequence and the promoter sequence can still be considered “operably linked” to the coding sequence. In fact, such sequences need not reside on the same contiguous DNA molecule (i.e. chromosome) and may still have interactions resulting in altered regulation.

A “PAM mutation” refers to one or more edits to a target sequence that removes, mutates, or otherwise renders inactive a PAM (i.e., protospacer adjacent motif) or spacer region in the target sequence.

A “promoter” or “promoter sequence” is a DNA regulatory region capable of binding RNA polymerase and initiating transcription of a polynucleotide or polypeptide coding sequence such as messenger RNA, ribosomal RNA, small nuclear or nucleolar RNA, guide RNA, or any kind of RNA. Promoters may be constitutive or inducible. A “pol II promoter” is a regulatory sequence that is bound by RNA polymerase II to catalyze the transcription of DNA.

As used herein the term “repair template” in the context of a CREATE fusion editing system employing a nickase-RT fusion enzyme refers to a nucleic acid (here, a ribonucleic acid) that is designed to serve as a template (including a desired edit) to be incorporated into target DNA via reverse transcriptase.

The term “RNA stability moiety” refers to a moiety, such as those listed infra in Table 1, appended to the 3′ end of the repair template in a CF editing cassette. The term “stabilized CF editing cassette” or “StCFEC” refers to a CF editing cassette comprising an RNA stability moiety at the 3′ end of the repair template.

As used herein the term “selectable marker” refers to a gene introduced into a cell, which confers a trait suitable for artificial selection. General use selectable markers are well-known to those of ordinary skill in the art. Drug selectable markers such as ampicillin/carbenicillin, kanamycin, chloramphenicol, nourscothricin N-acetyl transferase, erythromycin, tetracycline, gentamicin, bleomycin, streptomycin, puromycin, hygromycin, blasticidin, and G418 may be employed. In other embodiments, selectable markers include, but are not limited to human nerve growth factor receptor (detected with a MAb, such as described in U.S. Pat. No. 6,365,373); truncated human growth factor receptor (detected with MAb); mutant human dihydrofolate reductase (DHFR; fluorescent MTX substrate available); secreted alkaline phosphatase (SEAP; fluorescent substrate available); human thymidylate synthase (TS; confers resistance to anti-cancer agent fluorodeoxyuridine); human glutathione S-transferase alpha (GSTA1; conjugates glutathione to the stem cell selective alkylator busulfan; chemoprotective selectable marker in CD34+cells); CD24 cell surface antigen in hematopoietic stem cells; human CAD gene to confer resistance to N-phosphonacetyl-L-aspartate (PALA); human multi-drug resistance-1 (MDR-1; P-glycoprotein surface protein selectable by increased drug resistance or enriched by FACS); human CD25 (IL-2α; detectable by Mab-FITC); Methylguanine-DNA methyltransferase (MGMT; selectable by carmustine); rhamnose; and Cytidine deaminase (CD; selectable by Ara-C). “Selective medium” as used herein refers to cell growth medium to which has been added a chemical compound or biological moiety that selects for or against selectable markers.

The terms “target DNA sequence”, “target region”, “cellular target sequence”, or “genomic target locus” refer to any locus in vitro or in vivo, or in a nucleic acid (e.g., genome or episome) of a cell or population of cells, in which a change of at least one nucleotide is desired using a nucleic acid-guided nuclease editing system. The cellular target sequence can be a genomic locus or extrachromosomal locus. The target genomic DNA sequence comprises the edit region or edit locus.

A “vector” is any of a variety of nucleic acids that comprise a desired sequence or sequences to be delivered to and/or expressed in a cell. Vectors are typically composed of DNA, although RNA vectors are also available. Vectors include, but are not limited to, plasmids, fosmids, phagemids, virus genomes, BACs, YACs, PACs, synthetic chromosomes, and the like. As used herein, the phrase “engine vector” comprises a coding sequence for a nickase-RT fusion enzyme to be used in the CREATE fusion editing systems and methods of the present disclosure. As used herein the phrase “editing vector” comprises a repair template—including an alteration to the cellular target sequence that prevents nuclease binding at a PAM or spacer in the cellular target sequence after editing has taken place—covalently linked to a coding sequence for a gRNA. The editing vector may also and preferably does comprise a selectable marker and/or a barcode, and/or, as described herein, an RNA stabilization moiety. In some embodiments, the engine vector and editing vector may be combined; that is, all nickase-RT editing components may be found on a single vector. Further, the engine and editing vectors comprise control sequences operably linked to, e.g., the nickase-RT fusion enzyme coding sequence and the CF editing cassette.

### Nucleic Acid-Guided Nickase/Reverse Transcriptase Fusion Enzyme Genome Editing Generally

The compositions and methods described herein are a “twist on” or alternative to traditional nucleic acid-guided nuclease editing (i.e., RNA-guided nuclease editing or CRISPR editing) used to introduce desired edits to a population of cells; that is, the compositions and methods described herein employ a nucleic acid-guided nickase/reverse transcriptase fusion protein (“nickase-RT fusion”) as opposed to a nucleic acid-guided nuclease. The nickase-RT fusion employed herein differs from traditional CRISPR editing in that instead of initiating double-strand breaks in the target genome, the nickase initiates a nick in a single strand of the target genome. The fusion of the nickase to a reverse transcriptase eliminates the need for a repair template to be incorporated by homologous recombination; instead, the repair template is a nucleic acid—typically a ribonucleic acid—that serves as a template for the reverse transcription portion of the nickase-RT fusion. Utilization of a nickase-RT fusion incorporates the desired edit in the target genome at the RNA level rather than the DNA level. The nickase fused to a reverse transcriptase functions as the single-strand cutter (i.e., nickase)-having the specificity of a nucleic acid-guided nuclease—by first engaging the target DNA, then nicking a strand of the target DNA, followed by the annealing of the 3′ end of the CF editing cassette to the target DNA. The reverse transcriptase then copies the repair template to repair the target DNA thereby incorporating the desired edit into the target DNA. The present methods and compositions are drawn to stabilizing the 3′ end of the CF editing cassette with an RNA stabilization moiety, thereby creating a stabilized CF editing cassette or “StCFEC.”

Traditional nucleic acid-guided nuclease editing begins with a nucleic acid-guided nuclease complexing with an appropriate gRNA in a cell wherein the nucleic acid-guided nuclease can cut the genome of the cell at a desired location. The guide nucleic acid (i.e., gRNA) helps the nucleic acid-guided nuclease recognize and cut the DNA at a specific target sequence. By manipulating the nucleotide sequence of the guide nucleic acid, the nucleic acid-guided nuclease may be programmed to target any DNA sequence for cleavage as long as an appropriate protospacer adjacent motif (PAM) is nearby. In some CRISPR systems, the nucleic acid-guided nuclease editing system uses two separate guide nucleic acid molecules that combine to function as a guide nucleic acid, e.g., a CRISPR RNA (crRNA) and trans-activating CRISPR RNA (tracrRNA). In other CRISPR systems, the guide nucleic acid may be a single guide nucleic acid that includes both the crRNA and tracrRNA sequences. In general, a gRNA complexes with a compatible nucleic acid-guided nuclease that can then hybridize with a target sequence, thereby directing the nuclease to the target sequence. The nickase-RT fusions used in the present methods typically retain the PAM- and sequence-specificity of the nucleic acid-guided nucleases from which they are derived and, like nucleic acid-guided nucleases, complex with a gRNA.

A guide nucleic acid or gRNA comprises a guide sequence, where the guide sequence (as opposed to the scaffold sequence portion of the gRNA) is a polynucleotide sequence having sufficient complementarity with a target sequence to hybridize with the target sequence and direct sequence-specific binding of a complexed nucleic acid-guided nuclease to the target sequence. The degree of complementarity between a guide sequence and the corresponding target sequence, when optimally aligned using a suitable alignment algorithm, is about or more than about 50%, 60%, 75%, 80%, 85%. 90%, 95%. 97.5%. 99%, or more. Optimal alignment may be determined with the use of any suitable algorithm for aligning sequences. In some embodiments, a guide sequence is about or more than about 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. Preferably the guide sequence is 10-30 or 15-20 nucleotides long, or 15, 16, 17, 18, 19, or 20 nucleotides in length.

In the present methods and compositions, the gRNAs are provided as mRNAs or as sequences to be expressed from a CF editing cassette, optionally inserted into plasmid or vector and the gRNAs comprise both the guide sequence and the scaffold sequence as a single transcript. The gRNAs are engineered to target a desired target sequence by altering the guide sequence of the gRNA so that the guide sequence is complementary to a desired target DNA sequence, thereby allowing hybridization between the guide sequence and the target sequence. In general, to generate an edit in the target sequence, the gRNA/nickase-RT fusion complex binds to a target sequence as determined by the gRNA, and the nickase portion of the nickase-RT fusion recognizes a protospacer adjacent motif (PAM) sequence adjacent to the target DNA sequence. The target DNA sequence can be any polynucleotide endogenous or exogenous to a prokaryotic or eukaryotic cell, or in vitro. For example, the target DNA sequence can be a polynucleotide residing in the nucleus of a eukaryotic cell. A target DNA sequence can be a sequence encoding a gene product (e.g., a protein) or a non-coding sequence (e.g., a regulatory polynucleotide, an intron, a PAM, or “junk” DNA).

The gRNA is part of a CF editing cassette that also encodes the repair template which is copied by the reverse transcriptase portion of the nickase-RT fusion into the target DNA sequence.

The target DNA sequence is associated with a protospacer adjacent motif (PAM), which is a short nucleotide sequence recognized by the gRNA/nickase-RT fusion complex. The precise preferred PAM sequence and length requirements for different nucleic acid-guided nucleases vary; however, PAMs typically are 2-7 base-pair sequences adjacent or in proximity to the target sequence and, depending on the nuclease, can be 5′ or 3′ to the target sequence. Engineering of the PAM-interacting domain of a nickase-RT fusion may allow for alteration of PAM specificity, improve target site recognition fidelity, decrease target site recognition fidelity, or increase the versatility of a nickase-RT fusion enzyme.

The range of target DNA sequences that nickase-RT fusion enzymes can recognize is constrained by the need for a specific PAM to be located near the desired target sequence. As a result, it often can be difficult to target edits with the precision that is necessary for genome editing. It has been found that nickase-RT fusion enzymes can recognize some PAMs very well (e.g., canonical PAMs), and other PAMs less well or poorly (e.g., non-canonical PAMs). In certain embodiments and preferably, the editing of a target DNA sequence both introduces a desired DNA change to the cellular target sequence, e.g., the genomic DNA of a cell, and removes, mutates, or renders inactive a protospacer mutation (PAM) region in the cellular target sequence. Rendering the PAM at the cellular target sequence inactive precludes additional editing of the cell genome at that cellular target sequence, e.g., upon subsequent exposure to a nickase-RT fusion complexed with a gRNA in later rounds of editing.

As for the nickase-RT fusion component of the nickase-RT fusion editing system, a polynucleotide sequence encoding the nickase-RT fusion can be codon optimized for expression in particular cell types, such as archaeal, prokaryotic or eukaryotic cells. Eukaryotic cells can be yeast, fungi, algae, plant, animal, or human cells. Eukaryotic cells may be those of or derived from a particular organism, such as a mammal, including but not limited to human, mouse, rat, rabbit, dog, or non-human mammals including non-human primates. The choice of nickase-RT fusion to be employed depends on many factors, such as what type of edit is to be made in the target sequence and whether an appropriate PAM is located close to the desired target sequence. For information of MADzyme nickases, see U.S. Pat. Nos. 10,883,077; 11,053,485; and 11,085,030; and U.S. Ser. No. 17/200,089 and Ser. No. 17/200,110 filed 12 Mar. 2021; Ser. No. 17/463,498, filed 23 Aug. 2021; and Ser. No. 17/463,581, filed 1 Sep. 2021.

In addition to the gRNA and repair template, an editing cassette may comprise and preferably does comprise one or more primer sites used to amplify the CF editing cassette by using oligonucleotide primers; for example, if the primer sites flank one or more of the other components of the CF editing cassette.

In addition, the CF editing cassette may comprise a barcode. A barcode is a unique DNA sequence that corresponds to the repair template sequence such that the barcode can identify the edit made to the corresponding cellular target sequence. The barcode typically comprises four or more nucleotides. In some embodiments, the CF editing cassettes comprise a collection or library of gRNAs and corresponding repair templates representing, e.g., gene-wide or genome-wide libraries of gRNAs and repair templates. The library of CF editing cassettes is cloned into vector backbones where, e.g., each different repair template is associated with a different barcode.

Improved Nucleic Acid-Guided Nickase/Reverse Transcriptase Fusion Editing using 3′ Stabilized Repair Templates

The present disclosure provides compositions of matter, methods and instruments for nucleic acid-guided nickase/reverse transcriptase fusion (“nickase-RT fusion”) editing of live cells using an RNA stabilization moiety at the 3′ end of a CF editing cassette (i.e., an “StCFEC”). With the present compositions and methods, editing efficiency is improved using fusion proteins (i.e., the nickase-RT fusions) that retain certain characteristics of nucleic acid-directed nucleases-the binding specificity and ability to cleave one or more DNA strands in a targeted manner combined with reverse transcriptase activity, which uses a repair template so that a desired edit is incorporated into the target DNA sequence at the RNA level.

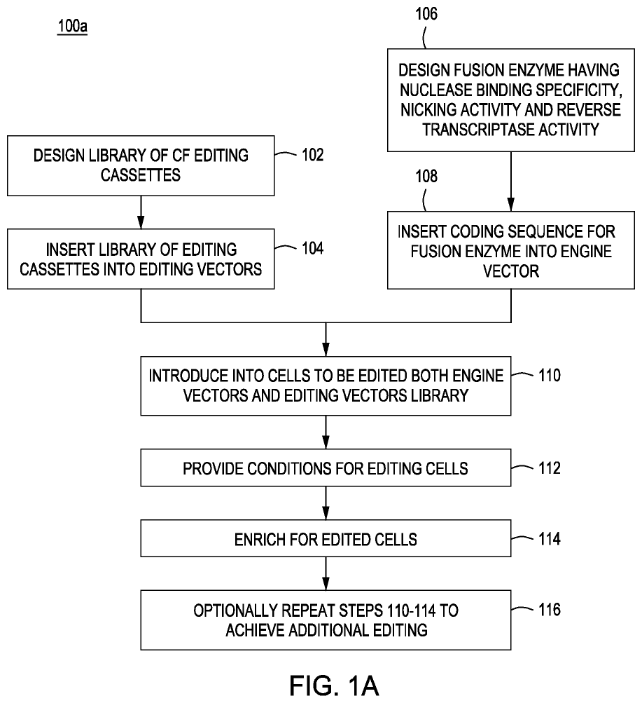

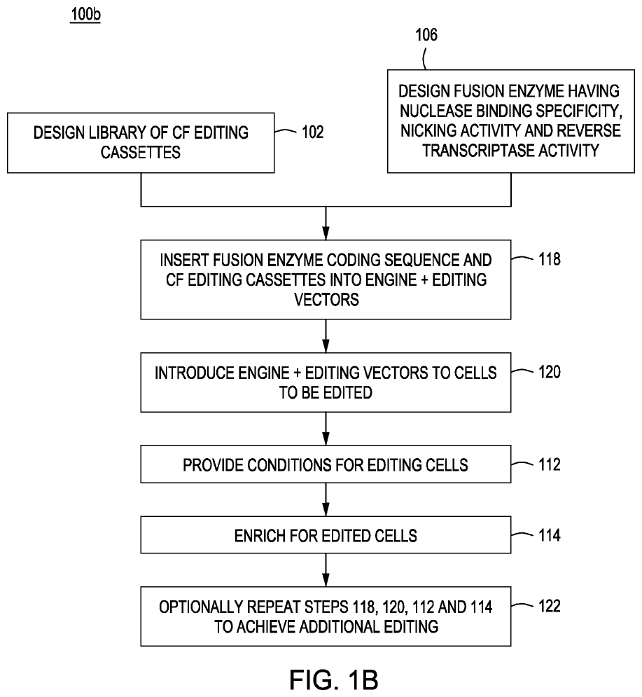

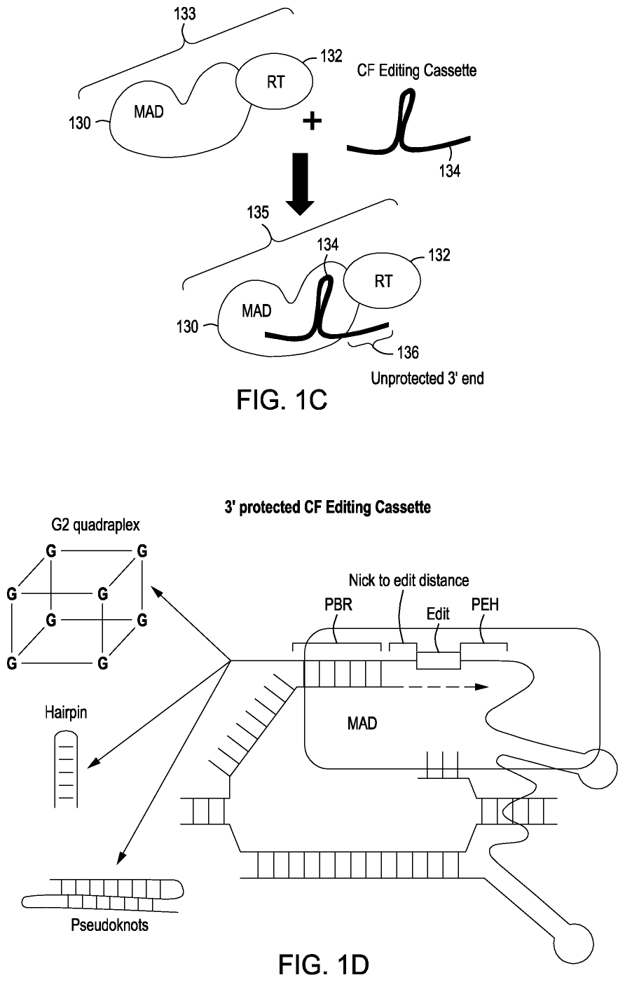

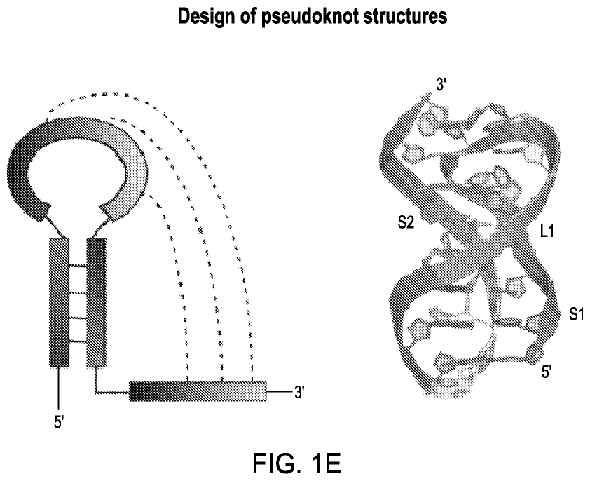

FIG. 1A is a simplified block diagram of an exemplary method 100a for editing live cells via nucleic acid-guided nickase/reverse transcriptase fusion (“nickase-RT fusion”) editing. Looking at FIG. 1A, method 100a begins by designing and synthesizing CF editing cassettes comprising a gRNA and a repair template comprising 3′ RNA stabilization sequences or StCFECs 102. As described above, each CF editing cassette comprises a gRNA sequence and a repair template-in the compositions and methods herein, the repair template comprises an RNA stabilization moiety on the 3′ end of the CF editing cassette sequence (“StCFEC”)—to be transcribed where the repair template comprises the desired target genome edits as well as a PAM or spacer mutation. Once the CF editing cassettes have been synthesized, the individual CF editing cassettes are amplified and inserted into a vector backbone, such as a lentiviral backbone, to create editing vectors 104. In addition, a nickase-RT fusion enzyme is designed 106. The nickase-RT fusion enzyme may be delivered to the cells as a coding sequence in a vector backbone (in some embodiments under the control of an inducible promoter) or the nickase-RT fusion enzyme may be delivered to the cells as a protein or protein complex. In method 100a, the nickase-RT fusion protein coding sequence is inserted into an engine vector 108 to be delivered to the cells. At step 110, the engine and editing vectors are introduced into the live cells.

A variety of delivery systems may be used to introduce (e.g., transform or transfect) nucleic acid-guided nickase fusion editing system components into a host cell 108. These delivery systems include the use of yeast systems, lipofection systems, microinjection systems, biolistic systems, virosomes, liposomes, immunoliposomes, polycations, lipid:nucleic acid conjugates, virions, artificial virions, viral vectors, electroporation, cell permeable peptides, nanoparticles, nanowires, exosomes. Alternatively, molecular trojan horse liposomes may be used to deliver nucleic acid-guided nuclease components across the blood brain barrier. Of particular interest is the use of electroporation, particularly flow-through electroporation (either as a stand-alone instrument or as a module in an automated multi-module system) as described in, e.g., U.S. Pat. Nos. 10,253,316; 10,329,559; 10,323,242; 10,421,959; 10,465,185; 10,519,437; and U.S. Ser. No. 16/666,964. filed 29 Oct. 2019. and Ser. No. 16/680,643, filed 12 Nov. 2019 all of which are herein incorporated by reference in their entirety.

Once transformed 110, the next step in method 100a is to provide conditions for nickase-RT fusion editing 112. “Providing conditions” includes incubation of the cells in appropriate medium and may also include providing conditions to induce transcription of an inducible promoter (e.g., adding antibiotics, increasing temperature) for transcription of one or both of the CF editing cassette and the nickase-RT fusion. Once editing is complete, the cells are allowed to recover and are preferably enriched for cells that have edited 114. Enrichment can be performed directly, such as via cells from the population that express a selectable marker, or by using surrogates, e.g., cell surface handles co-introduced with one or more components of the editing components and using cell sorting, e.g., using FACs (fluorescent activated cell sorting). At this point in method 100a, the cells can be characterized phenotypically or genotypically or optionally steps 110-114 may be repeated to make additional edits 116.

FIG. 1B is an alternative simplified block diagram of an exemplary method 100b for editing live cells via nickase-RT fusion editing. Looking at FIG. 1B, method 100b begins like method 100a by designing and synthesizing CF editing cassettes each comprising a gRNA and a repair template, wherein each repair template comprises an RNA stability moiety on the 3′ end as well as a desired target genome edit as well as a PAM or spacer mutation. In addition, a nickase-RT fusion enzyme is designed 106. As described above, the nickase-RT fusion protein may be delivered to the cells as a coding sequence in a vector backbone or the nickase-RT fusion protein may be delivered to the cells as a protein. In method 100b, the nickase-RT fusion protein is delivered to the cells via a coding sequence in a combined CF engine+editing vector 118, which at step 120, is introduced into the live cells. Again—as described above—there are a number of methods for introducing the combined CF engine+editing vector into the population of cells.

Following transformation 120, the next step in method 100b is to provide conditions for nucleic acid-guided nuclease editing 112. Again, “providing conditions” includes incubating the cells in an appropriate medium and may also include providing conditions to induce transcription of an inducible promoter (e.g., adding antibiotics, increasing temperature) for transcription of one or both of the CF editing cassette and the nickase-RT fusion. Once editing is complete, the cells are allowed to recover and are preferably enriched for cells that have edited 114. Again, enrichment can be performed directly, such as via cells from the population that express a selectable marker, or by using surrogates, e.g., cell surface handles co-introduced with one or more components of the editing components. At this point in method 100b, the cells can be characterized phenotypically or genotypically or optionally steps 118, 120, 112 and 114 may be repeated to make additional edits 122.

FIG. 1C is a simplified graphic depiction of a nickase-RT fusion and CF editing cassette. In FIG. 1C, there is seen the MAD nickase portion 130 and the reverse transcriptase portion 132 of the nickase-RT fusion 133, as well as the editing cassette 134. Once the nickase-RT fusion/CF editing cassette complex 135 is formed (e.g., 130+132+134), it can be seen that the 3′ end 136 of the CF editing cassette is unprotected and is vulnerable to degradation by 3′ exonucleases, whereas the 5′ portion of the CF editing cassette is protected by the nickase portion 130 of the nickase-RT fusion (130+132). The present methods and compositions are drawn to protecting the 3′ end of the CF editing cassette thereby forming, e.g., a CF editing cassette with a 3′ RNA stabilization moiety (i.e., a “StCFEC”).

FIG. 1D is a simplified graphic of a nickase-RT fusion and a CF editing cassette comprising a 3′ RNA stabilization moiety (StCFEC). The target DNA sequence that has been “unwound” and is bound to an StCFEC comprising from 3′to 5′: an RNA stabilization moiety (in FIG. 1D, a G2 quadraplex, an RNA hairpin structure, or an RNA pseudoknot), an optional linker region (not labeled), a primer binding region (PBR) which anneals to the genomic target region that is nicked, a variable nick-to-edit number of nucleotides, the region of the StCFEC comprising the desired edit and PAM edit, a region of post-edit homology (PEH), and the gRNA. The RNA stabilization moiety as shown here can be a G2 quadraplex or like structure, an RNA hairpin structure, a moiety such as an RNA pseudoknot structure (see Table 1, infra), or an exoribonuclease-resistant RNA (also described infra).

The linker region between the RNA stabilization moiety and the primer binding region can vary from 0 to 20 nucleotides, or from 2 to 15 nucleotides, or from 4-10 nucleotides. 5′ of the linker region is the a primer binding region (PBR) which anneals to the genomic target region that is nicked, followed by a nick-to-edit distance of 0 to 10 nucleotides in length and preferably 0 to 5 nucleotides in length. The edit region (edit) is the region of the StCFEC comprising the desired edit, as well as the one or more edits to the target sequence that removes, mutates, or otherwise renders inactive a PAM or spacer region in the target sequence. Following the region comprising the desired edit and the edit to the PAM is the post-edit homology region (PEH), which typically is from 3 to 20 nucleotides in length, or from 3 to 10 nucleotides in length. The post-edit homology region of the repair template optionally is contiguous or nearly contiguous with the guide sequence portion of the gRNA.

FIG. 1E are depictions of the generalized pseudoknot structure tested as a stabilization moiety (see Table 1, infra).

### Automated Cell Editing Instruments and Modules to Perform Nucleic Acid-Guided Nickase Fusion Editing in Cells

### One Embodiment of an Automated Cell Editing Instrument

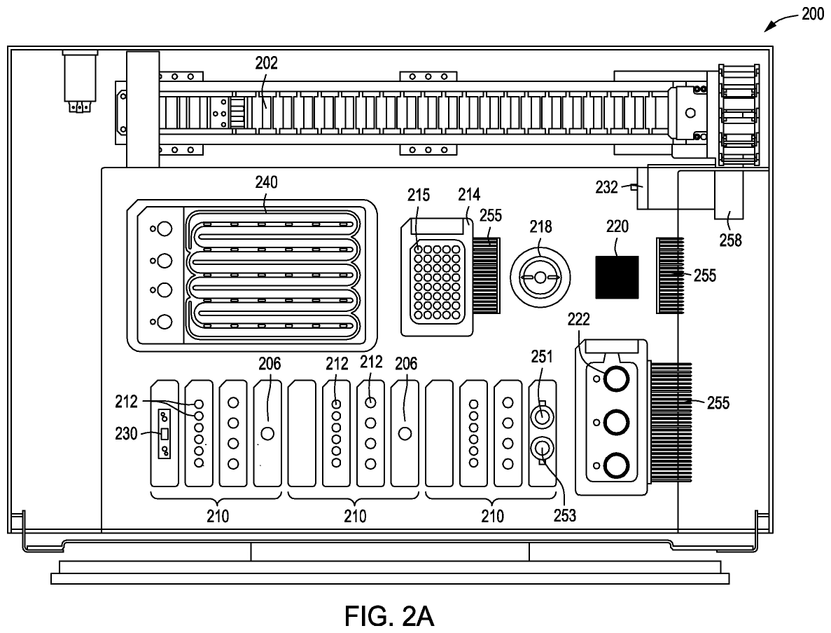

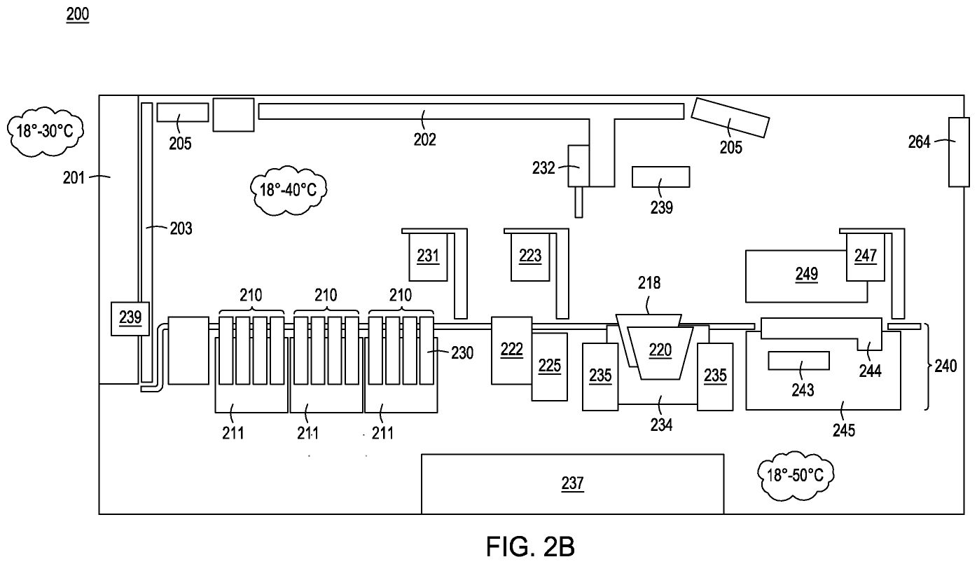

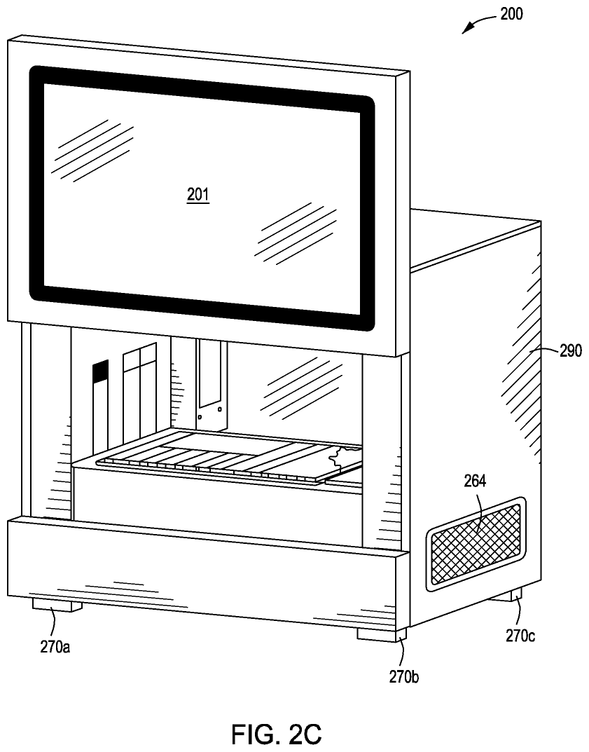

FIG. 2A depicts an exemplary automated multi-module cell processing instrument 200 to, e.g., perform targeted gene editing via a nickase-RT fusion in live cells. The instrument 200, for example, may be and preferably is designed as a stand-alone benchtop instrument for use within a laboratory environment. The instrument 200 may incorporate a mixture of reusable and disposable components for performing the various integrated processes in conducting automated genome cleavage and/or editing in cells without human intervention. Illustrated is a gantry 202, providing an automated mechanical motion system (actuator) (not shown) that supplies XYZ axis motion control to, e.g., an automated (i.e., robotic) liquid handling system 258 including, e.g., an air displacement pipettor 232 which allows for cell processing among multiple modules without human intervention. In some automated multi-module cell processing instruments, the air displacement pipettor 232 is moved by gantry 202 and the various modules and reagent cartridges remain stationary; however, in other embodiments, the liquid handling system 258 may stay stationary while the various modules and reagent cartridges are moved. Also included in the automated multi-module cell processing instrument 200 are reagent cartridges 210 (see, U.S. Pat. Nos. 10,376,889; 10,406,525; 10,478,822; 10,576,474; 10,639,637; 10,738,271; and 10,799,868) comprising reservoirs 212 and transformation module 230 (e.g., a flow-through electroporation (FTEP) device as described in U.S. Pat. Nos. 10,435,713; 10,443,074; and 10,851,389), as well as wash reservoirs 206, cell input reservoir 251 and cell output reservoir 253. The wash reservoirs 206 may be configured to accommodate large tubes, for example, wash solutions, or solutions that are used often throughout an iterative process. Although two of the reagent cartridges 210 comprise a wash reservoir 206 in FIG. 2A, the wash reservoirs instead could be included in a wash cartridge where the reagent and wash cartridges are separate cartridges. In such a case, the reagent cartridge and wash cartridge may be identical except for the consumables (reagents or other components contained within the various inserts) inserted therein.

In some implementations, the reagent cartridges 210 are disposable kits comprising reagents and cells for use in the automated multi-module cell processing/editing instrument 200. For example, a user may open and position each of the reagent cartridges 210 comprising various desired inserts and reagents within the chassis of the automated multi-module cell editing instrument 200 prior to activating cell processing. Further, each of the reagent cartridges 210 may be inserted into receptacles in the chassis having different temperature zones appropriate for the reagents contained therein.

Also illustrated in FIG. 2A is the robotic liquid handling system 258 including the gantry 202 and air displacement pipettor 232. In some examples, the robotic handling system 258 may include an automated liquid handling system such as those manufactured by Tecan Group Ltd. of Mannedorf, Switzerland, Hamilton Company of Reno, NV, USA (see, e.g., WO2018015544A1), or Beckman Coulter, Inc. of Fort Collins, CO, USA (see, e.g., US20160018427A1). Pipette tips 215 may be provided in a pipette transfer tip supply 214 for use with the air displacement pipettor 232. The robotic liquid handling system allows for the transfer of liquids between modules without human intervention.

Inserts or components of the reagent cartridges 210, in some implementations, are marked with machine-readable indicia (not shown), such as bar codes, for recognition by the robotic handling system 258. For example, the robotic liquid handling system 258 may scan one or more inserts within each of the reagent cartridges 210 to confirm contents. In other implementations, machine-readable indicia may be marked upon each reagent cartridge 210, and a processing system (not shown, but see element 237 of FIG. 2B) of the automated multi-module cell editing instrument 200 may identify a stored materials map based upon the machine-readable indicia. In the embodiment illustrated in FIG. 2A, a cell growth module comprises a cell growth vial 218 (for details, see U.S. Pat. Nos. 10,435,662; 10,433,031; 10,590,375; 10,717,959; and 10,883,095). Additionally seen is a tangential flow filtration (TFF) module 222 (for details, see U.S. Ser. No. 16/516,701 and Ser. No. 16/798,302). Also illustrated as part of the automated multi-module cell processing instrument 200 of FIG. 2A is a singulation module 240 (e.g., a solid wall isolation, incubation and normalization device (SWIIN device) is shown here and described in detail in U.S. Pat. Nos. 10,533,152; 10,633,626; 10,633,627; 10,647,958; 10,723,995; 10,801,008; 10,851,339; 10,954,485; 10,532,324; 10,625,212; 10,774,462; and 10,835,869), served by, e.g., robotic liquid handing system 258 and air displacement pipettor 232. Additionally seen is a selection module 220 which may employ magnet separation. Also note the placement of three heatsinks 255.

FIG. 2B is a simplified representation of the contents of the exemplary multi-module cell processing instrument 200 depicted in FIG. 2A. Cartridge-based source materials (such as in reagent cartridges 210), for example, may be positioned in designated areas on a deck of the instrument 200 for access by an air displacement pipettor 232 on gantry 202. The deck of the multi-module cell processing instrument 200 may include a protection sink (not shown) such that contaminants spilling, dripping, or overflowing from any of the modules of the instrument 200 are contained within a lip of the protection sink. Also seen are reagent cartridges 210, which are shown disposed with thermal assemblies 211 which can create temperature zones appropriate for different reagents in different regions. Note that one of the reagent cartridges also comprises a flow-through electroporation device 230 (FTEP), served by FTEP interface (e.g., manifold arm) and actuator 231. Also seen is TFF module 222 with adjacent thermal assembly 225, where the TFF module is served by TFF interface (e.g., manifold arm) and actuator 223. Thermal assemblies 225, 235, and 245 encompass thermal electric devices such as Peltier devices, as well as heatsinks, fans and coolers. The rotating growth vial 218 is within a growth module 234, where the growth module is served by two thermal assemblies 235. A selection module is seen at 220. Also seen is the SWIIN module 240, comprising a SWIIN cartridge 244, where the SWIIN module also comprises a thermal assembly 245, cooling grate 264, illumination 243 (in this embodiment, backlighting), evaporation and condensation control 249, and where the SWIIN module is served by SWIIN interface (e.g., manifold arm) and actuator 247. Also seen in this view is touch screen display 201, display actuator 203, illumination 205 (one on the side of multi-module cell processing instrument 200), and cameras 239 (one camera on either side of multi-module cell processing instrument 200). Finally, element 237 comprises electronics, such as a processor (237), circuit control boards, high-voltage amplifiers, power supplies, and power entry; as well as pneumatics, such as pumps, valves and sensors.

FIG. 2C illustrates a front perspective view of multi-module cell processing instrument 200 for use in as a benchtop version of the automated multi-module cell editing instrument 200. For example, a chassis 290 may have a width of about 24-48 inches, a height of about 24-48 inches and a depth of about 24-48 inches. Chassis 290 may be and preferably is designed to hold all modules and disposable supplies used in automated cell processing and to perform all processes required without human intervention; that is, chassis 290 is configured to provide an integrated, stand-alone automated multi-module cell processing instrument. As illustrated in FIG. 2C, chassis 290 includes touch screen display 201, cooling grate 264, which allows for air flow via an internal fan (not shown). The touch screen display provides information to a user regarding the processing status of the automated multi-module cell editing instrument 200 and accepts inputs from the user for conducting the cell processing. In this embodiment, the chassis 290 is lifted by adjustable feet 270a, 270b, 270c and 270d (feet 270a-270c are shown in this FIG. 2C). Adjustable feet 270a-270d, for example, allow for additional air flow beneath the chassis 290.

Inside the chassis 290, in some implementations, will be most or all of the components described in relation to FIGS. 2A and 2B, including the robotic liquid handling system disposed along a gantry, reagent cartridges 210 including a flow-through electroporation device, a rotating growth vial 218 in a cell growth module 234 (see FIG. 2B), a tangential flow filtration module 222, a SWIIN module 240 as well as interfaces and actuators for the various modules. In addition, chassis 290 houses control circuitry, liquid handling tubes, air pump controls, valves, sensors, thermal assemblies (e.g., heating and cooling units) and other control mechanisms. For examples of multi-module cell editing instruments, see U.S. Pat. Nos. 10,253,316; 10,329,559; 10,323,242; 10,421,959; 10,465,185; 10,519,437; 10,584,333; 10,584,334; 10,647,982; 10,689,645; 10,738,301; 10,738,663; 10,947,532; 10,894,958; 10,954,512; and 11,034,953, all of which are herein incorporated by reference in their entirety.

### Alterative Embodiment of an Automated Cell Editing Instrument

A bioreactor may be used to grow cells off-instrument or to allow for cell growth, editing and recovery on-instrument; e.g., as one module of a multi-module fully-automated closed instrument. Further, the bioreactor supports cell selection/enrichment, via expressed antibiotic markers in the growth process or via expressed antibodies coupled to magnetic beads and a magnet associated with the bioreactor. There are many bioreactors known in the art, including those described in, e.g., WO2019/046766; U.S. Pat. Nos. 10,699,519; 10,633,625; 10,577,576; 10,294,447; 10,240,117; 10,179,898; 10,370,629; and 9,175,259; and those available from Lonza Group Ltd. (Basel, Switzerland); Miltenyi Biotec (Bergisch Gladbach, Germany), Terumo BCT (Lakewood, CO, USA) and Sartorius GmbH (Gottingen, Germany).

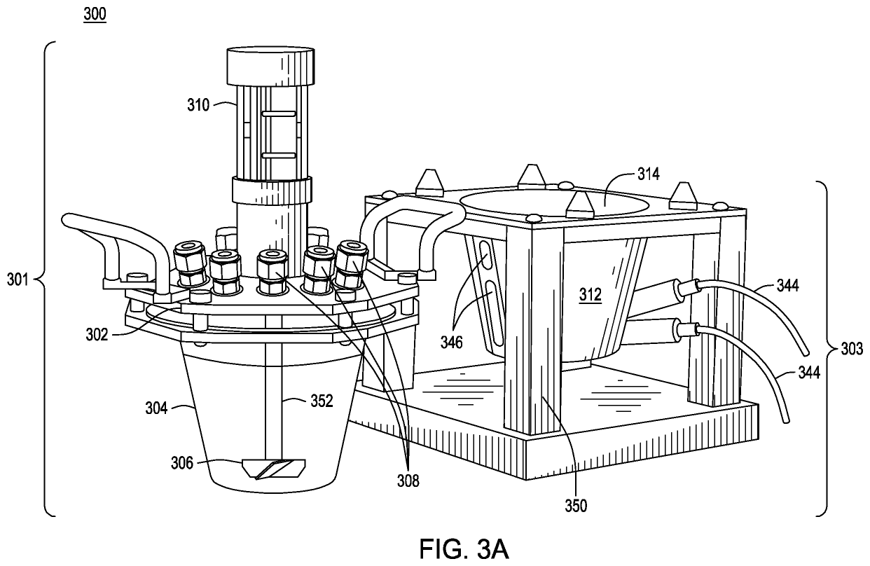

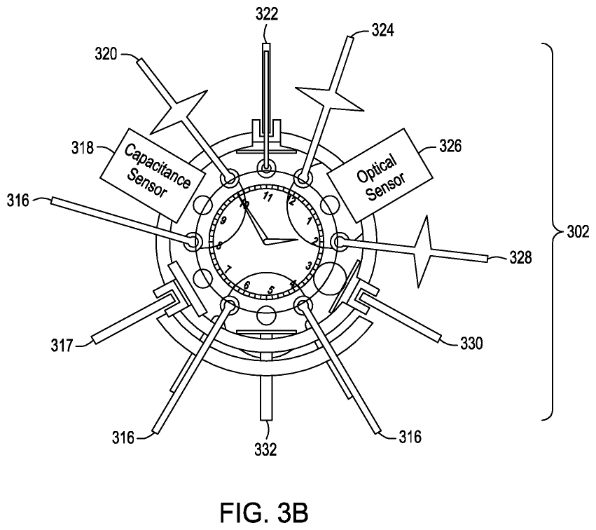

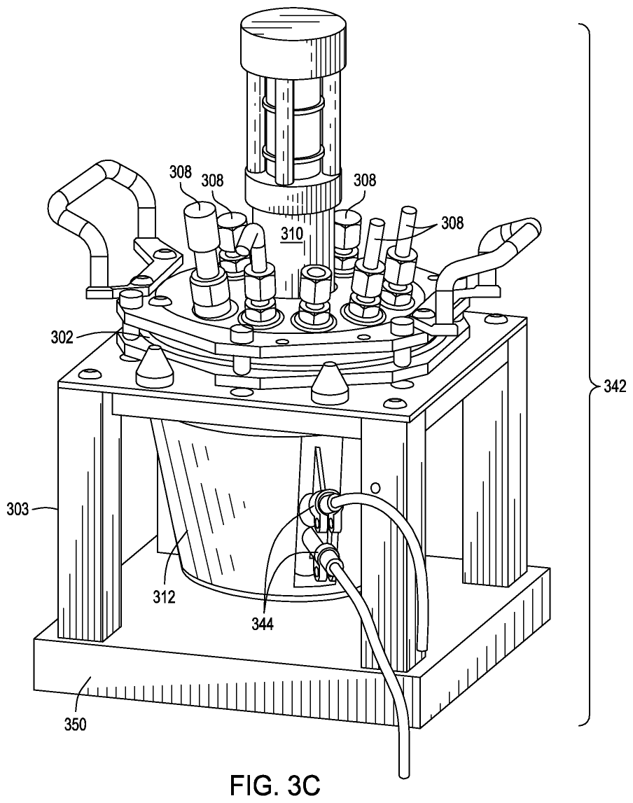

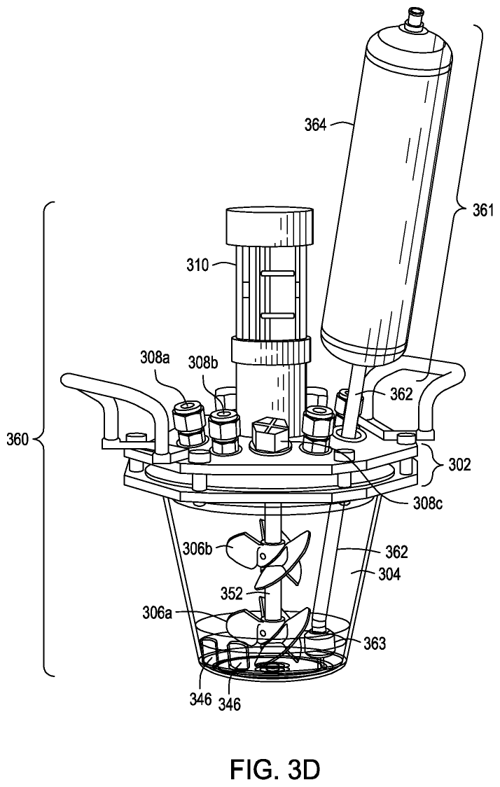

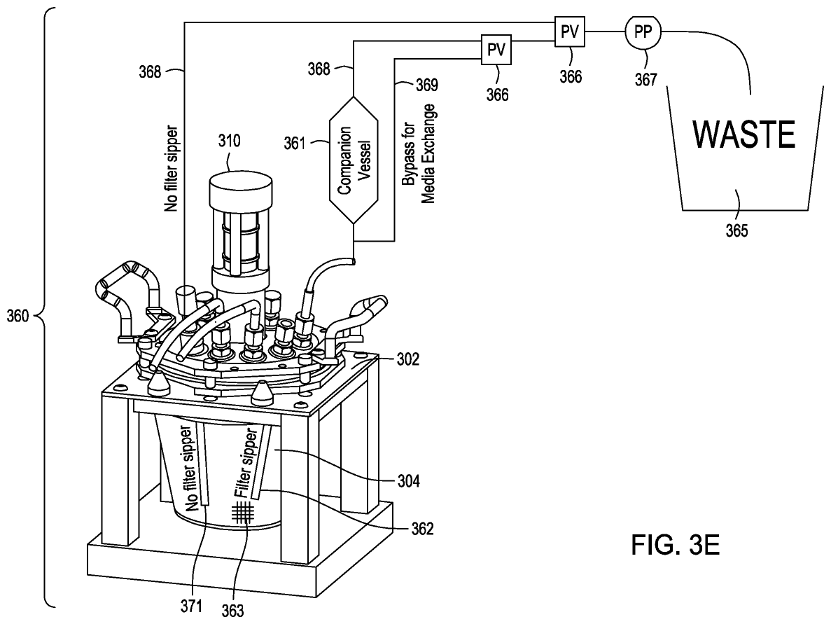

FIG. 3A shows one embodiment of a bioreactor assembly 300 suitable for cell growth, transfection, and editing in the automated multi-module cell processing instruments described herein. Unlike most bioreactors that are used to support fermentation or other processes with an eye to harvesting the products produced by organisms grown in the bioreactor, the present bioreactor (and the processes performed therein) is configured to grow cells, monitor cell growth (via, e.g., optical means or capacitance), passage cells, select cells, transfect cells, and support the growth and harvesting of edited cells. Bioreactor assembly 300 comprises cell growth vessel 301 comprising a main body 304 with a lid assembly 302 comprising ports 308, including a motor integration port 310 configured to accommodate a motor to drive impeller 306 via impeller shaft 352. The tapered shape of main body 304 of the growth vessel 301 along with, in some embodiments, dual impellers allows for working with a larger dynamic range of volumes, such as, e.g., up to 500 ml and as low as 100 ml for rapid sedimentation of the microcarriers.

Bioreactor assembly 300 further comprises bioreactor stand assembly 303 comprising a main body 312 and growth vessel holder 314 comprising a heat jacket or other heating means (not shown) into which the main body 304 of growth vessel 301 is disposed in operation. The main body 304 of growth vessel 301 is biocompatible and preferably transparent—in some embodiments, in the UV and IR range as well as the visible spectrum—so that the growing cells can be visualized by, e.g., cameras or sensors integrated into lid assembly 302 or through viewing apertures or slots 346 in the main body 312 of bioreactor stand assembly 303. Camera mounts are shown at 344.

Bioreactor assembly 300 supports growth of cells from a 500,000 cell input to a 10 billion cell output, or from a 1 million cell input to a 25 billion cell output, or from a 5 million cell input to a 50 billion cell output or combinations of these ranges depending on, e.g., the size of main body 304 of growth vessel 301, the medium used to grow the cells, the type and size and number of microcarriers used for growth (if microcarriers are used), and whether the cells are adherent or non-adherent. The bioreactor that comprises assembly 300 supports growth of both adherent and non-adherent cells, wherein adherent cells are typically grown of microcarriers as described in detail in U.S. Ser. No. 17/237,747, filed 24 Apr. 2021. Alternatively, another option for growing mammalian cells in the bioreactor described herein is growing single cells in suspension using a specialized medium such as that developed by ACCELLTA™ (Haifa, Israel). Cells grown in this medium must be adapted to this process over many cell passages; however, once adapted the cells can be grown to a density of >40 million cells/ml and expanded 50-100× in approximately a week, depending on cell type.

Main body 304 of growth vessel 301 preferably is manufactured by injection molding, as is, in some embodiments, impeller 306 and the impeller shaft 352. Impeller 306 also may be fabricated from stainless steel, metal, plastics or the polymers listed infra. Injection molding allows for flexibility in size and configuration and also allows for, e.g., volume markings to be added to the main body 304 of growth vessel 301. Additionally, material from which the main body 304 of growth vessel 301 is fabricated should be able to be cooled to about 4° C. or lower and heated to about 55° C. or higher to accommodate cell growth. Further, the material that is used to fabricate the vial preferably is able to withstand temperatures up to 55° C. without deformation. Suitable materials for main body 304 of growth vessel 301 include cyclic olefin copolymer (COC), glass, polyvinyl chloride, polyethylene, polyetheretherketone (PEEK), polypropylene, polycarbonate, poly(methyl) methacrylate (PMMA), polysulfone, poly(dimethylsiloxane), cyclo-olefin polymer (COP), and co-polymers of these and other polymers. Preferred materials include polypropylene, polycarbonate, or polystyrene. The material used for fabrication may depend on the cell type to be grown, transfected and edited, and be conducive to growth of both adherent and non-adherent cells and workflows involving microcarrier-based transfection. The main body 304 of growth vessel 301 may be reusable or, alternatively, may be manufactured and configured for a single use. In one embodiment, main body 304 of growth vessel 301 may support cell culture volumes of 25 ml to 500 ml, but may be scaled up to support cell culture volumes of up to 3 L.

The bioreactor stand assembly comprises a stand or frame 350, a main body 312 which holds the growth vessel 301 during operation. The stand/frame 350 and main body 312 are fabricated from stainless steel, other metals, or polymer/plastics. The bioreactor stand assembly main body further comprises a heat jacket (not seen in FIG. 3A) to maintain the growth vessel main body 304—and thus the cell culture—at a desired temperature. Additionally, the stand assembly can host a set of sensors and cameras (camera mounts are shown at 344) to monitor cell culture.

FIG. 3B depicts a top-down view of one embodiment of vessel lid assembly 302. Growth vessel lid assembly 302 is configured to be air-tight, providing a sealed, sterile environment for cell growth, transfection and editing as well as to provide biosafety in a closed system. Vessel lid assembly 302 and the main body 304 of growth vessel 301 (not shown here but on FIG. 3A) can be reversibly sealed via fasteners such as screws, or permanently sealed using biocompatible glues or ultrasonic welding. Vessel lid assembly 302 in some embodiments is fabricated from stainless steel such as S316L stainless steel but may also be fabricated from metals, other polymers (such as those listed supra) or plastics. As seen in this FIG. 3B—as well as in FIG. 3A—vessel lid assembly 302 comprises a number of different ports to accommodate liquid addition and removal; gas addition and removal; for insertion of sensors to monitor culture parameters (described in more detail infra); to accommodate one or more cameras or other optical sensors; to provide access to the main body 304 of growth vessel 301 by, e.g., a liquid handling device; and to accommodate a motor for motor integration to drive one or more impellers 306. Exemplary ports depicted in FIG. 3B include three liquid-in ports 316 (at 4 o'clock, 6 o'clock and 8 o'clock), one liquid-out port 322 (at 11 o'clock), a capacitance sensor 318 (at 9 o'clock), one “gas in” port 324 (at 12 o'clock), one “gas out” port 320 (at 10 o'clock), an optical sensor 326 (at 1 o'clock), a rupture disc 328 at 2 o'clock, two self-sealing ports 317, 330 (at 7 o'clock and 3 o'clock) to provide access to the main body 304 of growth vessel 301; and (a temperature probe 332 (at 5 o'clock) (note that the clock face is canted in this FIG. 3B).

The ports shown in vessel lid assembly 302 in this FIG. 3B are exemplary only and it should be apparent to one of ordinary skill in the art given the present disclosure that, e.g., a single liquid-in port 316 could be used to accommodate addition of all liquids to the cell culture rather than having a liquid-in port for each different liquid added to the cell culture. Similarly, there may be more than one gas-in port 324, such as one for each gas, e.g., O₂, CO₂ that may be added. In addition, although a temperature probe 332 is shown, a temperature probe alternatively may be located on the outside of vessel holder 314 of bioreactor stand assembly separate from or integrated into heater jacket (314, 302 not seen in this FIG. 3B). One or more self-sealing ports 317, 330, if present, allow access to the main body 304 of growth vessel 301 for, e.g., a pipette, syringe, or other liquid delivery system via a gantry (not shown). As shown in FIG. 3A, additionally there may be a motor integration port 310 to drive the impeller(s), although other configurations of growth vessel 301 may alternatively integrate the motor drive at the bottom of the main body 304 of growth vessel 301. Growth vessel lid assembly 302 may also comprise a camera port for viewing and monitoring the cells.

Additional sensors include those that detect dissolved O₂ concentration, dissolved CO₂ concentration, culture pH, lactate concentration, glucose concentration, biomass, and optical density. The sensors may use optical (e.g., fluorescence detection), electrochemical, or capacitance sensing and either be reusable or configured and fabricated for single-use. Sensors appropriate for use in the bioreactor are available from Omega Engineering (Norwalk, CT, USA); PreSens Precision Sensing (Regensburg, Germany); C-CIT Sensors AG (Waedenswil, Switzerland), and ABER Instruments Ltd. (Alexandria, VA, USA). In one embodiment, optical density is measured using a reflective optical density sensor to facilitate sterilization, improve dynamic range and simplify mechanical assembly. The rupture disc, if present, provides safety in a pressurized environment, and is programmed to rupture if a threshold pressure is exceeded in growth vessel. If the cell culture in the growth vessel is a culture of adherent cells, microcarriers may be used as described in U.S. Ser. No. 17/237,747, filed 24 Apr. 2021. In such an instance, the liquid-out port may comprise a filter such as a stainless steel or plastic (e.g., polyvinylidene difluoride (PVDF), nylon, polypropylene, polybutylene, acetal, polyethylene, or polyamide) filter or frit to prevent microcarriers from being drawn out of the culture during, e.g., medium exchange, but to allow dead cells to be withdrawn from the vessel. Additionally, a liquid port may comprise a filter sipper to allow cells that have been dissociated from microcarriers to be drawn into the cell corral while leaving spent microcarriers in main body of the growth vessel. The microcarriers used for initial cell growth can be nanoporous (where pore sizes are typically <20 nm in size), microporous (with pores between >20 nm to <1 μm in size), or macroporous (with pores between >1 μm in size, e.g. 20 μm) and the microcarriers are typically 50-200 μm in diameter; thus the pore size of the filter or frit in the liquid-out port will differ depending on microcarrier size.

The microcarriers used for cell growth depend on cell type and desired cell numbers, and typically include a coating of a natural or synthetic extracellular matrix or cell adhesion promoters (e.g., antibodies to cell surface proteins or poly-L-lysine) to promote cell growth and adherence. Microcarriers for cell culture are widely commercially available from, e.g., Millipore Sigma, (St. Louis, MO, USA); ThermoFisher Scientific (Waltham, MA, USA); Pall Corp. (Port Washington, NY, USA); GE Life Sciences (Marlborough, MA, USA); and Corning Life Sciences (Tewkesbury, MA, USA). As for the extracellular matrix, natural matrices include collagen, fibrin and vitronectin (available, e.g., from ESBio, Alameda, CA, USA), and synthetic matrices include MATRIGEL® (Corning Life Sciences, Tewkesbury, MA, USA). GELTREX™ (ThermoFisher Scientific, Waltham, MA, USA), CULTREX® (Trevigen, Gaithersburg, MD, USA), biomemetic hydrogels available from Cellendes (Tubingen, Germany); and tissue-specific extracellular matrices available from Xylyx (Brooklyn, NY, USA); further, denovoMatrix (Dresden, Germany) offers screenMATRIX™, a tool that facilitates rapid testing of a large variety of cell microenvironments (e.g., extracellular matrices) for optimizing growth of the cells of interest.

FIG. 3C is a side perspective view of the assembled bioreactor 342 without sensors mounted in ports 308. Seen are vessel lid assembly 302, bioreactor stand assembly 303, bioreactor stand main body 312 into which the main body of growth vessel 301 (not seen in this FIG. 3C) is inserted. Also present are two camera mounts 344, motor integration port 310 and base 350.

FIG. 3D shows the embodiment of a bioreactor/cell corral assembly 360, comprising the bioreactor assembly 300 (not shown in this FIG. 3D) for cell growth, transfection, and editing described in FIG. 3A and further comprising a cell corral 361. Bioreactor assembly comprises a growth vessel comprising tapered a main body 304 with a lid assembly 302 comprising ports 308a, 308b, and 308c, including a motor integration port 310 driving impellers 306a, 306b via impeller shaft 352, as well as two viewing ports 346. Cell corral 361 comprises a main body 364, end caps, where the end cap proximal the bioreactor assembly 300 is coupled to a filter sipper 362 comprising a filter portion 363 disposed within the main body 304 of the bioreactor assembly 300 (not shown in this FIG. 3D). The filter sipper is disposed within the main body 304 of the bioreactor assembly 300 but does not reach to the bottom surface of the bioreactor assembly 300 to leave a “dead volume” for spent microcarriers to settle while cells are removed from the growth vessel 301 into the cell corral 361. The cell corral may or may not comprise a temperature or CO₂ probe, and may or not be enclosed within an insulated jacket.

The cell corral 361, like the main body 304 of growth vessel is fabricated from any biocompatible material such as polycarbonate, cyclic olefin copolymer (COC), glass, polyvinyl chloride, polyethylene, polyetheretherketone (PEEK), polypropylene, poly(methyl methacrylate (PMMA)), polysulfone, poly(dimethylsiloxane), cyclo-olefin polymer (COP), and co-polymers of these and other polymers. Likewise, the end caps are fabricated from a biocompatible material such as polycarbonate, cyclic olefin copolymer (COC), glass, polyvinyl chloride, polyethylene, polyetheretherketone (PEEK), polypropylene, poly(methyl methacrylate (PMMA)), polysulfone, poly(dimethylsiloxane), cyclo-olefin polymer (COP), and co-polymers of these and other polymers. The cell corral may be coupled to or integrated with one or more devices, such as a flow cell where an aliquot of the cell culture can be counted. Additionally, the cell corral may comprise additional liquid ports for adding medium, other reagents, and/or fresh microcarriers to the cells in the cell corral. The volume of the main body 364 of the cell corral 361 may be from 25 to 3000 mL, or from 250 to 1000 mL, or from 450 to 500 mL.

In operation, the bioreactor/cell corral assembly 360 comprising the bioreactor assembly 300 (not shown in this FIG. 3D) and cell corral 361 grows, passages, transfects, and supports editing and further growth of mammalian cells (note, the bioreactor stand assembly is not shown in this FIG. 3D). Cells are transferred to the growth vessel comprising medium and microcarriers. The cells are allowed to adhere to the microcarries. Approximately 2,000,000 microcarriers (e.g., laminin-521 coated polystyrene with enhanced attachment surface treatment) are used for the initial culture of approximately 20 million cells to where there are approximately 50 cells per microcarrier. The cells are grown until there are approximately 500 cells per microcarrier. For medium exchange, the microcarriers comprising the cells are allowed to settle and spent medium is aspirated via a sipper filter, wherein the filter has a mesh small enough to exclude the microcarriers. The mesh size of the filter will depend on the size of the microcarriers and cells present but typically is from 50 to 500 μm, or from 70 to 200 μm, or from 80 to 110 μm. For passaging the cells, the microcarriers are allowed to settle and spent medium is removed from the growth vessel, and phosphobuffered saline or another wash agent is added to the growth vessel to wash the cells on the microcarriers. Optionally, the microcarriers are allowed to settle once again, and some of the wash agent is removed. At this point, the cells are dissociated from the microcarriers. Dissociation may be accomplished by, e.g., bubbling gas or air through the wash agent in the growth vessel, by increasing the impeller speed and/or direction, by enzymatic action (via, e.g., trypsin), or by a combination of these methods. In one embodiment, a chemical agent such as the RelesR™ reagent (STEMCELL Technologies Canada INC., Vancouver, BC, Canada) is added to the microcarriers in the remaining wash agent for a period of time required to dissociate most of the cells from the microcarriers, such as from 1 to 60 minutes, or from 3 to 25 minutes, or from 5 to 10 minutes. Once enough time has passed to dissociate the cells, cell growth medium is added to the growth vessel to stop the enzymatic reaction.

Once again, the now-spent microcarriers are allowed to settle to the bottom of the growth vessel and the cells are aspirated through a filter sipper into the cell corral 361. The growth vessel is configured to allow for a “dead volume” of 2 mL to 200 mL, or 6 mL to 50 mL, or 8 mL to 12 mL below which the filter sipper does not aspirate medium to ensure the settled spent microcarriers are not transported to the filter sipper during fluid exchanges. Once the cells are aspirated from the bioreactor vessel leaving the “dead volume” of medium and spent microcarriers, the spent microcarriers are aspirated through a non-filter sipper into waste. The spent microcarriers (and the bioreactor vessel) are diluted in phosphobuffered saline or other buffer one or more times, wherein the wash agent and spent microcarriers continue to be aspirated via the non-filter sipper leaving a clean bioreactor vessel. After washing, fresh microcarriers or RBMCs and fresh medium are dispensed into the bioreactor vessel and the cells in the cell corral are dispensed back into the bioreactor vessel for another round of passaging or for transfection and editing, respectively.

FIG. 3E depicts a bioreactor and bioreactor/cell corral assembly 360 comprising a growth vessel, with a main body 304, lid assembly 302 comprising a motor integration port 310, a filter sipper 362 comprising a filter 363 and a no-filter sipper 371. Also seen is a cell corral 361, fluid lines 368 from the cell corral through pinch valve 366, and a line 369 for medium exchange also connected to a pinch valve 366. The no-filter sipper 368 also runs through a pinch valve 366 to waste 365. Also seen is a peristaltic pump 367. For more detailed information on bioreactors and cell corrals, see U.S. Ser. No. 17/239,540, filed 24 Apr. 2021.

### Exemplary Embodiments for Delivery of Reagent Bundles to Mammalian Cells in a Bioreactor

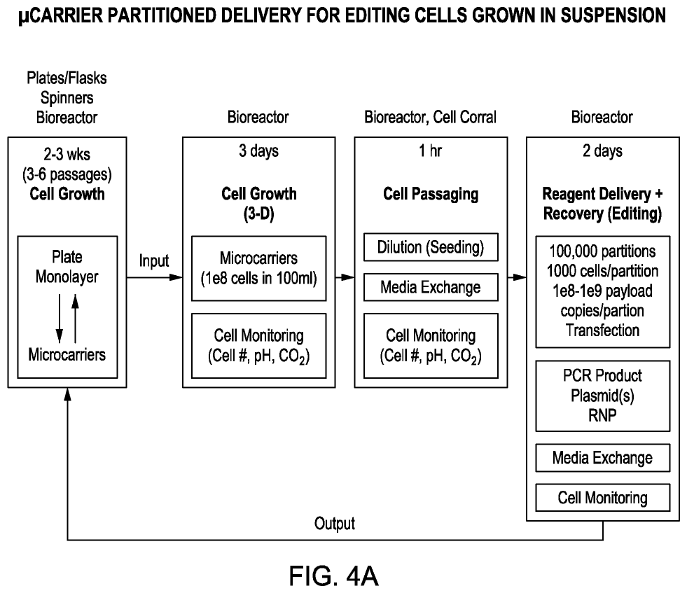

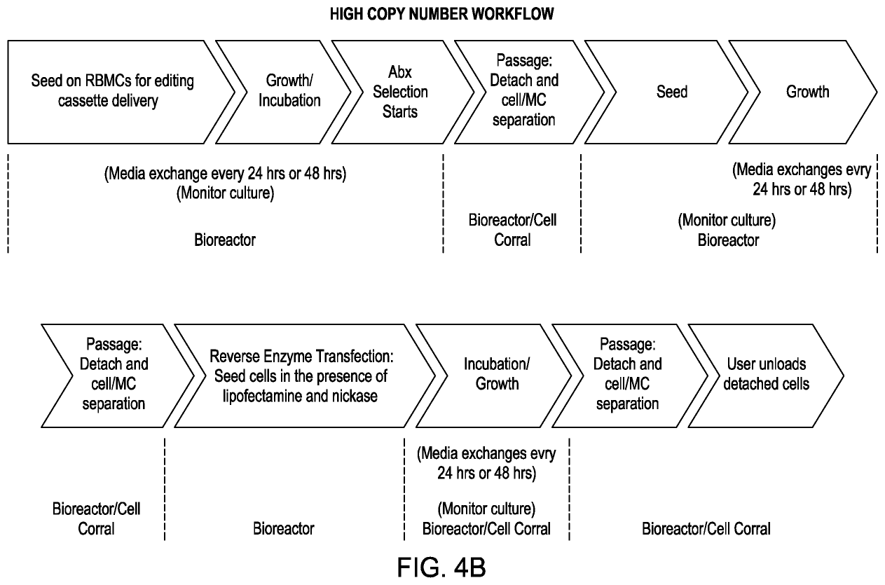

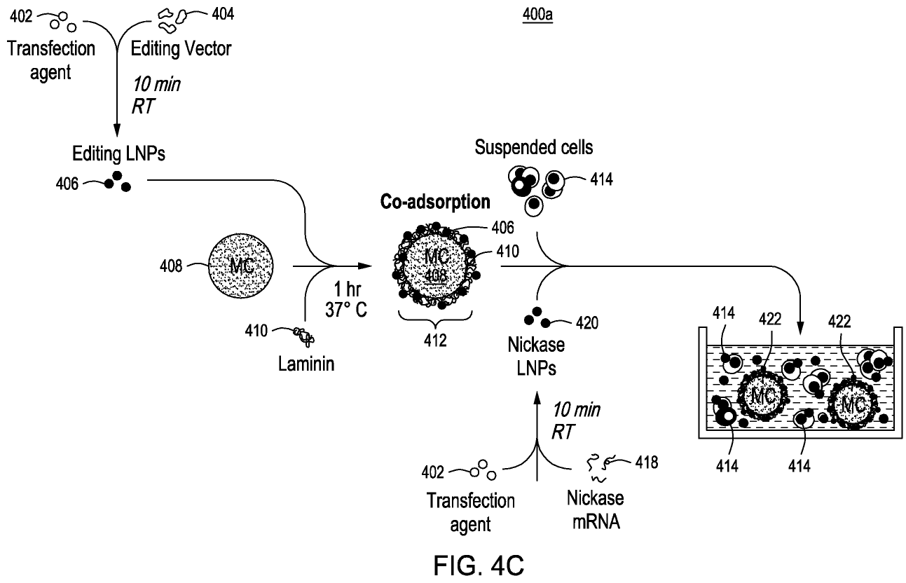

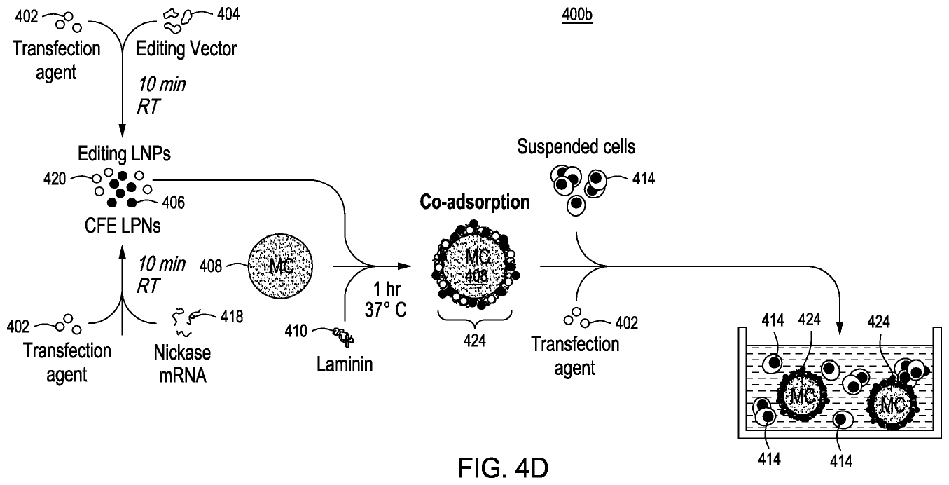

FIG. 4A depicts an exemplary workflow employing microcarrier-partitioned delivery for editing mammalian cells grown in suspension where the cells are co-localized on reagent bundle microcarriers (“RBMCs”) comprising the nickase-RT editing components to be transfected into the cell. In a first step, the cells to be edited are grown for several passages, e.g., off instrument, to assure cell health. The cells may be grown in 2D culture, in 3D culture (if the cells are viable when grown in or adapted to 3D culture) or on microcarriers. This initial cell growth typically takes place off the automated instrument. If necessary, the cells are dissociated and added to medium in the bioreactor comprising cell growth medium such as MEM, DMEM, RPMI, or, for stem cells, mTeSR™Plus serum-free, feeder-free cell culture medium (STEMCELL Technologies Canada INC., Vancouver, BC, Canada) and cell growth microcarriers. If the cells are grown initially on microcarriers, the microcarriers are transferred to the bioreactor comprising cell growth medium such as mTeSR™Plus serum-free, feeder-free cell culture medium (STEMCELL Technologies Canada INC., Vancouver, BC, Canada) and additional microcarriers. Approximately 1e7 or 1e8 cells are transferred to the cell growth module on the automated instrument for growth.

In parallel with the off-instrument cell growth, reagent bundle microcarriers (RBMCs) are manufactured, also off-instrument. The present description provides depictions of two exemplary methods where several steps involve manufacturing RBMCs (see FIGS. 4C and 4D) that may be used to edit the cells in the modules and automated instruments described herein.

The cells are grown in 3D culture on microcarriers in the bioreactor for, e.g., three to four days or until a desired number of cells, e.g., 1e8, cells are present. Note that all processes in this FIG. 4A may take place in the bioreactor and cell corral. During this growth cycle, the cells are monitored for cell number, pH, and optionally other parameters. As described above, cell growth monitoring can be performed by imaging, for example, by allowing the microcarriers to settle and imaging the bottom of the bioreactor. Alliteratively, an aliquot of the culture may be removed and ran through a separate flow cell, e.g., in a separate module, for imaging. For example, the cell corral, in addition to being integrated with the bioreactor vessel, may be integrated with a flow cell or other device for cell counting where an aliquot of the cell culture in the cell corral may be removed and counted in the flow cell.

In another alternative, the cells may express a fluorescent protein and fluorescence in the cell culture is measured or fluorescent dye may be used to stain cells, particularly live cells. This microcarrier-based workflow can be performed in the bioreactor and cell corral with most if not all steps performed in the same device; thus, several bioreactors and cell corrals may be deployed in parallel for two to many samples simultaneously. In yet another alternative, permittivity or capacitance is used to monitor cell coverage on the microcarriers. In yet another embodiment, an aliquot of cells may be removed from the bioreactor or cell corral and transported out of the instrument and manually counted on a commercial cell counter (i.e., Thermofisher Countess, Waltham, MA, USA).

The microcarriers used for initial cell growth can be nonporous (where pore sizes are typically <20 nm in size), microporous (with pores between >20 nm to <1 μm in size), or macroporous (with pores between >1 μm in size, e.g. 20 μm). In microcarrier culture, cells grow as monolayers on the surface of nonporous or microporous microcarriers, which are typically spherical in morphology; alternatively, the cells grow on the surface and as multilayers in the pores of macroporous microcarriers. The microcarriers preferably have a density slightly greater than that of the culture medium to facilitate easy separation of cells and medium for, e.g., medium exchange and imaging and passaging; yet the density of the microcarriers is also sufficiently low to allow complete suspension of the microcarriers at a minimum stirring or babbling rate. Maintaining a low stirring or bubbling rate is preferred so as to avoid hydrodynamic damage to the cells.

The microcarriers used for cell growth depend on cell type and desired cell numbers, and typically include a coating of a natural or synthetic extracellular matrix or cell adhesion promoters (e.g., antibodies to cell surface proteins or poly-L-lysine) to promote cell growth and adherence. Microcarriers for cell culture are widely commercially available from, e.g., Millipore Sigma, (St. Louis, MO, USA); Thermo Fisher (Waltham, MA, USA); Pall Corp. (Port Washington, NY, USA); GE Life Sciences (Marlborough, MA, USA); and Corning Life Sciences (Tewkesbury, MA, USA). As for the extracellular matrix, natural matrices include collagen, fibrin and vitronectin (available, e.g., from ESBio, Alameda, CA, USA), and synthetic matrices include Matrigel® (Corning Life Sciences, Tewkesbury, MA, USA). Geltrex™ (Thermo Fisher Scientific, Waltham, MA, USA), Cultrex® (Trevigen, Gaithersburg, MD, USA), biomemetic hydrogels available from Cellendes (Tubingen, Germany); and tissue-specific extracellular matrices available from Xylyx (Brooklyn, NY, USA); further, denovoMatrix (Dresden, Germany) offers screenMATRIX™, a tool that facilitates rapid testing of a large variety of cell microenvironments (e.g., extracellular matrices) for optimizing growth of the cells of interest.

Following cell growth, passaging is performed by, e.g., stopping the impeller rotation or bubbling action in the bioreactor and allowing the microcarriers to settle. In one method, the cells are removed from the microcarriers using enzymes such as collagenase, trypsin or pronase, or by non-enzymatic methods including EDTA or other chelating chemicals, and once removed from the carriers, medium is added to dilute the enzyme to inhibit enzymatic action. The dissociation procedures relating to the cell corral are described in detail infra. Once medium is added, then the cells are separated from the microcarriers by allowing the microcarriers to settle and aspirating the cells via a filtered sipper into the cell corral. The cells then may be optionally dissociated from one another via a filter, sieve or by bubbling or other agitation in the cell corral. Next, microcarriers comprising the manufactured reagent bundles (reagent bundle microcarrier microcarriers or RBMCs) and the dissociated cells are combined in an appropriate medium in the growth vessel. Alternatively, instead of removing cells from the cell growth microcarriers and re-seeding on RBMCs, the cells may be transferred from the cell growth microcarriers to RBMCs via microcarrier bridge passaging either in the growth vessel in a reduced volume or in the cell corral. Bridge passaging involves allowing a new microcarrier (e.g. an RBMC) to come into physical contact with a cell-laden microcarrier, such that cells on the latter microcarrier can migrate to the RBMC.

RBMCs are not prepared on-instrument but are pre-manufactured. The microcarriers used for reagent bundles may be microporous microcarriers, which, due to the plethora of micropores, can carry a larger reagent payload per carrier diameter than nonporous or macroporous microcarriers. Preferred microcarriers are microporous, to provide increased surface area for reagent delivery, and functionalized on the surface so as to be able to bind reagents. Preferred microcarriers for RBMCs include Pierce™ Streptavidin UltraLink™ Resin, a cross-linked polyacrylamide carrier functionalized with streptavidin comprising a pore size of 50 to 100 nm; Pierce™ NeutrAvidin™ Plus UltraLink™ Resin, cross-linked polyacrylamide carrier functionalized with avidin comprising a pore size of 50 to 100 nm; and UltraLink™ Hydrazide Resin, a cross-linked polyacrylamide carrier functionalized with hydrazine comprising a pore size of 50 to 100 nm, all available from Thermo Fisher (Waltham, MA, USA); cross-linked agarose resins with alkyne, azide, photo-cleavable azide and disulfide surface functional groups available from Click Chemistry Tools (Scottsdale, AZ, USA); Sepharose™ Resin, cross-linked agarose with amine, carboxyl, carbodiimide, N-hydroxysuccinimide (NHS), and epoxy surface functional groups available from GE Health (Chicago, IL, USA).

The microcarriers are loaded with amplified CF editing cassettes or amplified CF editing plasmids, engine plasmids, nickase-RT fusion enzyme, nickase-RT fusion mRNAs or ribonucleoproteins (RNPs) depending on, e.g., the functionalized group, via, e.g., via chemical or photo linkage or depending on a surface coating on the microcarrier, if present. RBMCs are prepared by 1) partitioning and amplifying a single copy of an editing cassette to produce clonal copies in an RBMC, or by 2) pooling and amplifying editing cassettes, followed by dividing the editing cassettes into sub-pools and “pulling down” the amplified editing cassettes with microcarriers comprising nucleic acids specific to and complementary to unique sequences on the editing cassettes. The step of sub-pooling acts to “de-multiplex” the editing cassette pool, thereby increasing the efficiency and specificity of the “pull down” process. De-multiplexing thus allows for amplification and error correction of the editing cassettes to be performed in bulk followed by efficient loading of clonal copies of the editing cassettes onto a microcarrier.

FIG. 4B depicts an exemplary option for growing, passaging, transfecting and editing iPSCs (induced pluripotent stem cells), where there is sequential delivery of clonal high copy number (HCN) RBMCs—i.e., lipid nanoparticle-coated microcarriers, where each microcarrier is coated with many copies of delivery vehicles (CF editing cassettes or CF editing vectors) carrying a single clonal editing cassette—followed by bulk enzyme delivery. Note that the bioreactors and cell corrals described supra may be used for all processes. Following the workflow of FIG. 1B, first cells are seeded on the RBMCs to deliver clonal copies of CF editing cassettes to the cells. Again, the RBMCs are typically fabricated or manufactured off-instrument. The cells are allowed to grow and after 24-48 hours, medium is exchanged for medium containing antibiotics to select for cells that have been transfected. The cells are passaged, re-seeded and grown again, and then passaged and re-seeded, this time onto microcarriers comprising lipofectamine with the nickase-RT fusion enzyme provided as a coding sequence under the control of a promoter, or as a protein on the surface of a microcarrier. As an alternative, the nickase-RT fusion enzyme may be provided in bulk in solution. The nickase-RT fusion enzyme is taken up by the cells on the microcarriers, and the cells are incubated and allowed to grow. Medium is exchanged as needed and the cells are detached from the microcarriers for subsequent growth and analysis.

An alternative exemplary option for the method shown in FIG. 4B comprises the steps of growing, passaging, transfecting and editing iPSCs. In this embodiment, there is simultaneous delivery of CF editing cassette RBMCs (i.e., reagent bundle lipid nanoparticle-coated microcarriers) where each microcarrier is coated with many copies of the CF editing cassettes or CF editing vectors carrying a single clonal the CF editing cassette and nickase-RT fusion enzyme (e.g., as a coding sequence under the control of a promoter therefor, as a ribonucleoprotein complex, or as a protein). Again, the RBMCs are typically fabricated or manufactured off-instrument. Note that the integrated instrument described infra may be used for all processes. As with the workflow shown in FIG. 4B, first cells are seeded on microcarriers to grow. The cells are then passaged, detached, re-seeded, grown and detached again to increase cell number, with medium exchanged every 24-48 or 24-72 hours as needed. Following detachment, the cells are seeded on RBMCs for clonal delivery of the editing cassette and enzyme in a co-transfection reaction. Following transfection, the cells grown for 24-48 hours after which medium is exchanged for medium containing antibiotics for selection. The cells are selected and passaged, re-seeded and grown again. Medium is exchanged as needed and the cells are detached from the microcarriers for subsequent growth and analysis.

FIGS. 4C and 4D depict alternative methods for populating microcarriers with a lipofectamine/CF editing cassette payload and cells. In the method 400a shown in FIG. 4C at top left, lipofectamine 402 and editing cassette payloads 404 are combined and editing LNPs (lipofectamine nucleic acid payloads) 406 are formed in solution. In parallel, microcarriers 408 (“MCs”) are combined with a coating such as laminin 521 410 to foster adsorption and cell attachment. The laminin 521-coated microcarriers are then combined with the editing LNPs 406 to form partially-loaded microcarriers 412. The processes of forming RBMCs (i.e., the partially-loaded microcarriers 412 comprising the editing LNPs 406) to this point are typically performed off-instrument. In parallel and typically off-instrument, nickase LNPs 420 are formed by combining lipofectamine 402 and nickase mRNA 418. The nickase LNPs 420 are combined with the partially-loaded microcarriers 412 and adsorb onto the partially-loaded microcarriers 412 to form fully-loaded RBMCs 422 comprising both the editing LNPs 406 and the nickase LNPs 420. At this point, the mammalian cells 414 have been grown and passaged in the bioreactor and cell corral several to many times. The cells 414 populate the fully-loaded RBMCs 422, where the cells 414 then take up (i.e., are transfected by) the editing LNPs 406 and the nickase LNPs 420, a process that may take several hours up to several days. At the end of the transfection process, transfected mammalian cells reside on the surface of the fully-loaded microcarriers 422.

As an alternative to the method 400a shown in FIG. 4C, FIG. 4D depicts method 400b which features simultaneous adsorption of the editing LNPs and the nickase LNPs. Again, lipofectamine 402 and editing vector payloads 404 are combined where editing LNPs (lipofectamine nucleic acid payloads) 406 are formed in solution. In parallel, nickase LNPs 420 are formed by combining lipofectamine 402 and nickase mRNA 418. Also in parallel, microcarriers 408 are combined with a coating such as laminin 521 410 to foster adsorption and cell attachment. The laminin 521-coated microcarriers are simultaneously combined with both the editing LNPs 406 and the nickase LNPs 420 to form fully-loaded microcarriers 424 where both the editing LNPs 406 and the nickase LNPs 420 co-adsorb onto the surface of the laminin-coated microcarriers. The processes of forming RBMCs (i.e., the fully-loaded microcarriers 424 comprising both the editing LNPs 406 and the nickase-RT fusion LNPs 420) to this point are typically performed off-instrument.

At this point, the fully-loaded microcarriers 424 comprising the editing LNPs 406 and the nickase-RT fusion LNPs 420 are added to medium in the bioreactor comprising the mammalian cells 414 to be transfected, optionally with additional lipofect reagent 402. The mammalian cells 414 have been grown and passaged in the bioreactor and cell corral one to many times. The cells 414 populate the fully-loaded RBMCs 424, where the cells 414 then take op (i.e., are transfected by) the editing LNPs 406 and the nickase-RT fusion LNPs 420, a process that may take several hours up to several days. At the end of the transfection process, transfected mammalian cells reside on the surface of the fully-loaded microcarriers 424. In these exemplary methods, nickase-RT fusion mRNAs are used to form the nickase-RT fusion LNPs; however, the nickase-RT enzymes may be loaded on to form LNPs, or CF editing cassettes and nickase-RT fusion enzymes may be loaded in the form of ribonucleoproteins (RNPs) on the LNPs. For additional details on microcarriers and RBMCs, please see U.S. Ser. No. 17/239,540, filed 24 Apr. 2021.

### Use of the Automated Multi-Module Cell Processing Instrument

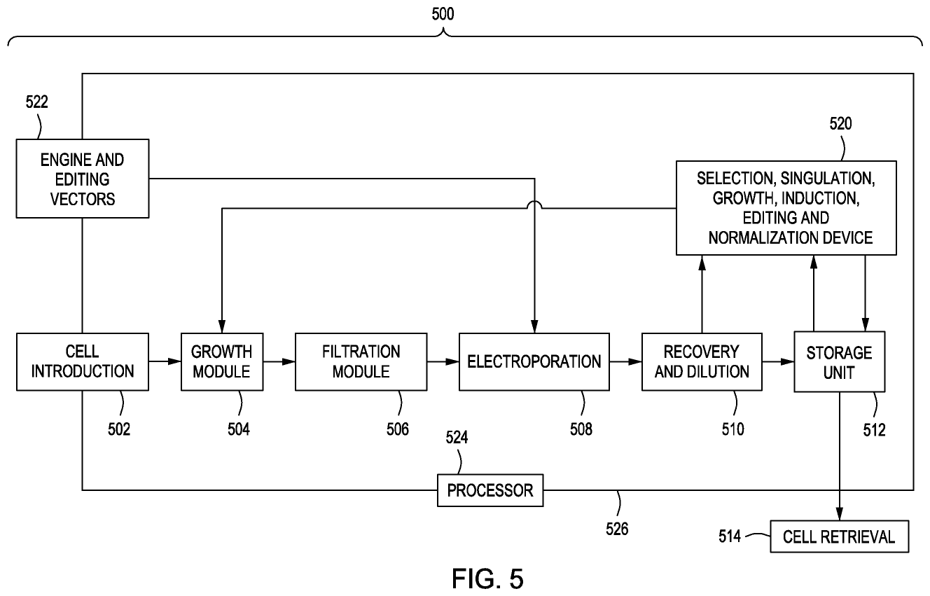

FIG. 5 illustrates an embodiment of a multi-module cell processing instrument. This embodiment depicts an exemplary system that performs recursive gene nickase-RT fusion editing on a cell population. The cell processing instrument 500 may include a housing 526, a reservoir for storing cells to be transformed or transfected 502. and a cell growth module (comprising, e.g., a rotating growth vial) 504. The cells to be transformed are transferred from a reservoir 502 to the cell growth module 504 to be cultured until the cells hit a target OD. Once the cells hit the target OD, the growth module may cool or freeze the cells for later processing or transfer the cells to a cell concentration (e.g., filtration) module 506 where the cells are subjected to buffer exchange and rendered electrocompetent and the volume of the cells may be reduced substantially. Once the cells have been concentrated to an appropriate volume, the cells are transferred to electroporation device 508 or other transformation module. In addition to the reservoir for storing cells 502, the multi-module cell processing instrument includes a reservoir for storing the engine and editing vectors or engine+editing vectors or vectors and nickase-RT enzymes to be introduced into the electrocompetent cell population 522. The vectors are transferred to the electroporation device 508, which already contains the cell culture grown to a target OD. In the electroporation device 508, the nucleic acids (or nucleic acids and proteins) are electroporated into the cells. Following electroporation, the cells are transferred into an optional recovery and dilution module 510, where the cells recover briefly post-transformation.

After recovery, the cells may be transferred to a storage module 512, where the cells can be stored at, e.g., 4° C. or −20° C. for later processing, or the cells may be diluted and transferred to a selection/singulation/growth/induction/editing/normalization (or, e.g., SWIIN) module 520. In the SWIIN 520, the cells are arrayed such that there is an average of one to twenty or fifty or so cells per microwell. The arrayed cells may be in selection medium to select for cells that have been transformed or transfected with the editing vector(s). Once singulated, the cells grow through 2-50 doublings and establish colonies. Once colonies are established, editing is induced by providing conditions (e.g., temperature, addition of an inducing or repressing chemical) to induce editing. Editing is then initiated and allowed to proceed, the cells are allowed to grow to terminal size (e.g., normalization of the colonies) in the microwells and then are treated to conditions that care the editing vector from this round. Once cured, the cells can be flushed out of the microwells and pooled, then transferred to the storage (or recovery) unit 512 or can be transferred back to the growth module 504 for another round of editing. In between pooling and transfer to a growth module, there typically is one or more additional steps, such as cell recovery, medium exchange (rendering the cells electrocompetent), cell concentration (typically concurrently with medium exchange by, e.g., filtration).

Note that the selection/singulation/growth/induction/editing/normalization and curing modules may be the same module, where all processes are performed in, e.g., a solid wall device, or selection and/or dilution may take place in a separate vessel before the cells are transferred to the solid wall singulation/growth/induction/editing/normalization/editing module (or e.g., SWIIN) 520. Similarly, the cells may be pooled after normalization, transferred to a separate vessel, and cured in the separate vessel. As an alternative to singulation in, e.g., a solid wall device, the transformed cells may be grown in—and editing can be induced in—bulk liquid (see, e.g., U.S. Ser. No. 16/540,767, filed 14 Aug. 2019 and Ser. No. 16/545,097, filed 20 Aug. 2019). Once the putatively-edited cells are pooled, they may be subjected to another round of editing, beginning with growth, cell concentration and treatment to render electrocompetent, and transformation by yet another repair template in another editing cassette via the electroporation module 508.

In electroporation device 508, the cells selected from the first round of editing are transformed by a second set of editing vectors and the cycle is repeated until the cells have been transformed and edited by a desired number of, e.g., CF editing cassettes. The multi-module cell processing instrument exemplified in FIG. 5 is controlled by a processor 524 configured to operate the instrument based on user input or is controlled by one or more scripts including at least one script associated with the reagent cartridge. The processor 524 may control the timing, duration, and temperature of various processes, the dispensing of reagents, and other operations of the various modules of the instrument 500. For example, a script or the processor may control the dispensing of cells, reagents, vectors, and editing cassettes; which editing cassettes are used for cell editing and in what order; the time, temperature and other conditions used in the recovery and expression module, the wavelength at which OD is read in the cell growth module, the target OD to which the cells are grown, and the target time at which the cells will reach the target OD. In addition, the processor may be programmed to notify a user (e.g., via an application) as to the progress of the cells in the automated multi-module cell processing instrument.

It should be apparent to one of ordinary skill in the art given the present disclosure that the process described may be recursive and multiplexed; that is, cells may go through the workflow described in relation to FIG. 5, then the resulting edited culture may go through another (or several or many) rounds of additional editing (e.g., recursive editing) with different CF editing cassettes. For example, the cells from round 1 of editing may be diluted and an aliquot of the edited cells edited by CF editing cassette A may be combined with CF editing cassette B, an aliquot of the edited cells edited by CF editing cassette A may be combined with CF editing cassette C, an aliquot of the edited cells edited by CF editing cassette A may be combined with CF editing cassette D, and so on for a second round of editing. After round two, an aliquot of each of the double-edited cells may be subjected to a third round of editing, where, e.g., aliquots of each of the AB-, AC-, AD-CF edited cells are combined with additional editing cassettes, such as CF editing cassettes X, Y, and Z. That is, double-edited cells AB may be combined with and edited by CF editing cassettes X, Y, and Z to produce triple-edited edited cells ABX, ABY, and ABZ; double-edited cells AC may be combined with and edited by CF editing cassettes X, Y, and Z to produce triple-edited cells ACX, ACY, and ACZ; and double-edited cells AD may be combined with and edited by CF editing cassettes X. Y, and Z to produce triple-edited cells ADX, ADY, and ADZ, and so on. In this process, many permutations and combinations of edits can be executed, leading to very diverse cell populations and cell libraries.

In any recursive process, it is advantageous to “cure” the editing vectors comprising the CF editing cassette. “Curing” is a process in which one or more CF editing vectors used in the prior round of editing is eliminated from the transformed cells. Curing can be accomplished by, e.g., cleaving the editing vector(s) using a curing plasmid thereby rendering the editing vectors nonfunctional; diluting the editing vector(s) in the cell population via cell growth (that is, the more growth cycles the cells go through, the fewer daughter cells will retain the editing vector(s)), or by, e.g., utilizing a heat-sensitive origin of replication on the editing vector. The conditions for curing will depend on the mechanism used for curing; that is, in this example, how the curing plasmid cleaves the editing vector. For additional information on curing, see, e.g., U.S. Pat. Nos. 10,837,021 and 11,053,507; and U.S. Ser. No. 17,353,282, filed 21 Jun. 2021; and Ser. No. 17/300,518, filed 27 Jul. 2021.

### EXAMPLES

The following examples are put forth so as to provide those of ordinary skill in the art with a complete disclosure and description of how to make and use the present invention and are not intended to limit the scope of what the inventor regards as his invention, nor are they intended to represent or imply that the experiments below are all of or the only experiments performed. It will be appreciated by persons skilled in the art that numerous variations and/or modifications may be made to the invention as shown in the specific aspects without departing from the spirit or scope of the invention as broadly described. The present aspects are, therefore, to be considered in all respects as illustrative and not restrictive.

### Example I: GFP to BEP Conversion Assay

A GFP to BFP reporter cell line was created using mammalian cells with a stably integrated genomic copy of the GFP gene (HEK293T-GFP). These cell lines enabled phenotypic detection of genomic edits of different classes (NHEJ, HDR, no edit) by various different mechanisms, including flow cytometry, fluorescent cell imaging, and genotypic detection by sequencing of the genome-integrated GFP gene. Lack of editing, or perfect repair of cut events in the GFP gene, result in cells that remain GFP-positive. Cut events that are repaired by the Non-Homologous End-Joining (NHEJ) pathway often result in nucleotide insertion or deletion events (indels), resulting in frame-shift mutations in the coding sequence that cause loss of GFP gene expression and fluorescence. Cut events that are repaired by the Homology-Directed Repair (HDR) pathway, using the GFP to BFP HDR donor as a repair template, result in conversion of the cell fluorescence profile from that of GFP to that of BFP.

### Example II: CREATE Fusion Editing

The CREATE fusion editing system is a live cell editing system that uses a nickase-RT fusion protein (e.g., MAD2007 nickase and others, see U.S. Pat. Nos, 10,883,077; 11,053,485; and 11,085,030; and U.S. Ser. No. 17/200,089 and Ser. No. 17/200,110 filed 12 Mar. 2021; Ser. No. 17/463,498. filed 23 Aug. 2021; and Ser. No. 17/463,581. filed 1 Sep. 2021) fused to a peptide with reverse transcriptase activity along with a nucleic acid encoding a gRNA/repair template (i.e., CF editing cassette) comprising a region complementary to a target region of a nucleic acid in one or more cells, which comprises a mutation of at least one nucleotide relative to the target region in the one or more cells and a protospacer adjacent motif (PAM) mutation.

In a first design, a nickase enzyme derived from the MAD2007 nuclease (see U.S. Pat. Nos. 9,982,279 and 10,337,028), e.g., MAD7 nickase (see U.S. Pat. No. 10,883,077), was fused to an engineered reverse transcriptase (RT) on the C-terminus and cloned downstream of a CMV promoter. In this instance, the RT used was derived from Moloney Murine Leukemia Virus (MMLV).

gRNAs and repair templates (CF editing cassettes) were designed that were complementary to a single region proximal to the EGFP-to-BFP editing site. The repair template on the 3′ end included a region of 13 bp comprising the TY-to-SH edit and a second region of 13 bp that was complementary to the nicked EGFP DNA sequence. This allowed the nicked genomic DNA to anneal to the 3′ end of the repair template which can then be extended by the reverse transcriptase to incorporate the edit in the genome. A second gRNA and repair template (CF editing cassette) targeted a region in the EGFP DNA sequence that is 86 bp upstream of the edit site. This CF editing cassette was designed such that it enables the nickase to cut the opposite strand relative to the other CF editing cassette. Both of these CF editing cassettes were cloned downstream of a U6 promoter. A poly-T sequence was also included that terminates the transcription of the CF editing cassette.

The plasmids were transformed into NEB stable E. coli (Ipswich, NY, USA) and grown overnight in 25 mL LB cultures. The following day the plasmids were purified from E. coli using the Qiagen Midi Prep kit (Venlo, Netherlands). The purified plasmid was then RNase A (ThermoFisher, Waltham, MA, USA) treated and re-purified using the DNA Clean and Concentrator kit (Zymo, Irvine, CA, USA).

HEK293T cells were cultured in DMEM medium which was supplemented with 10% FBS and 1× Penicillin and Streptomycin. 100 ng of total DNA (50 ng of gRNA plasmid and 50 ng of CFE plasmids) was mixed with 1 μl of PolyFect (Qiagen, Venlo, Netherlands) in 25 μl of OptiMEM in a 96 well plate. The complex was incubated for 10 minutes and then 20,000 HEK293T cells resuspended in 100 μl of DMEM were added to the mixture. The resulting mixture was then incubated for 80 hours at 37 C and 5% CO₂.

The cells were harvested from flat bottom 96 well plates using TrypLE Express reagent (ThermoFisher, Waltham, MA, USA) and transferred to v-bottom 96-well plate. The plate was then spun down at 500 g for 5 minutes. The TrypLE solution was then aspirated and the cell pellet was resuspended in FACS buffer (1×PBS. 1% FBS, 1 mM EDTA and 0.5% BSA). The GFP+, BFP+and RFP+ cells were then analyzed on the Attune NxT flow cytometer and the data was analyzed on FlowJo software.

The RFP+BFP+ cells that were identified were indicative of the proportion of enriched cells that have undergone a precise or imprecise editing process. BFP+ cells indicate cells that have undergone successful editing process and express BFP. The GFP− cells indicate cells that have been imprecisely edited, leading to disruption of the GFP open reading frame and loss of expression.

In this exemplary experiment, the edit is positioned roughly 5′ in the repair template and 3′ of the edit is a region complementary to the nicked genome, although the intended edit could also be present further within the region homologous to the nicked genome. A nickase-RT fusion enzyme (MAD2007 nickase) created a nick in the target site and the nicked DNA annealed to its complementary sequence on the 3′ end of the repair template. The reverse transcriptase portion of the nickase-RT fusion then extended the DNA, thereby incorporating the intended edit directly in the genome.

The effectiveness of the CREATE fusion editing system in GFP+HEK293T cells was then tested. In the assay system devised, a successful precise edit resulted in a BFP+ cell whereas imprecisely edited cells turned the cell both BFP and GFP negative. A CF editing cassette in combination with CFE2.1 or CFE2.2 gave ˜40-45% BFP+ cells indicating that almost half the cell population has undergone successful editing (data not shown), The GFP− cells are ˜10% of the population. The use of a second nicking editing cassette, as described in Liu, et al., Nature, 576 (7785): 149-157 (2019) did not increase the precision edit rate any further; in fact, it significantly increased the imprecisely edited, GFP-negative cell population and the editing rate was lower.

Previous literature has shown that double nicks on opposite strands (<90 bp away) do result in a double strand break which tend to be repaired via NHEJ resulting in imprecise insertions or deletions. Overall, the results indicated that CREATE fusion editing predominantly yielded precisely edited cells and the imprecisely edited cells proportion is much lower (data not shown).

An enrichment handle, specifically a fluorescent reporter (in this case, red fluorescent protein or RFP) linked to nuclease expression was included in this experimentation as a proxy for cells receiving the editing machinery. When only the RFP-positive cells were analyzed (computational enrichment) after 3-4 cell divisions, up to 75% of the cells were BFP+ when tested with CF editing cassettes (data not shown), indicating uptake or expression-linked reporters can be used to enrich for a population of cells with higher rates of CREATE fusion editing system-mediated gene editing. In fact, the combined use of CREATE fusion editing and the described enrichment methods resulted in a significantly improved rate of intended edits (data not shown).

### Example III: CREATE Fusion Editing With CF Editing Cassette

CREATE fusion editing was carried out in mammalian cells using a CF editing cassette having an intended edit to the native sequence and an edit that disrupts nuclease cleavage at this site. Briefly, lentiviral vectors were produced using the following protocol: 1000 ng of Lentiviral transfer plasmid containing the editing cassettes along with 1500 ng of Lentiviral Packaging plasmids (ViraSafe Lentivirus Packaging System Cell BioLabs) were transfected into HEK293T cells using Lipofectamine LTX in 6-well plates. Media containing the lentivirus was collected 72 hrs post transfection. Two clones of a lentiviral CF editing cassette design were chosen, and an empty lentiviral backbone was included as negative control.

The day before the transduction, 200,000 HEK293T cells were seeded in six well plates. Different volumes of CF editing cassette lentivirus (10 to 1000 μl) were added to HEK293T cells in 6-well plates along with 10 μg/ml of Polybrene. 48 hours after transduction, media with 15 μg/ml of Blasticidin was added to the wells. Cells were maintained in selection for one week. Following selection, the well with lowest number of surviving cells was selected for future experiments (<5% cells).

The experimental constructs or wild-type SpCas9 were electroporated into HEK293T cells using the Neon Transfection System (Thermo Fisher Scientific, Waltham, MA, USA). Briefly, 400 ng of total plasmid DNA was mixed with 100,000 cells in Buffer R in a total of 15 μl volume. The 10 μl Neon tip was used to electroporate cells using 2 pulses of 20 ms and 1150 v. Cells were analyzed on the flow cytometer 80 hrs post electroporation. Unenriched editing rates of up to 15% were achieved from single copy delivery of an editing cassette (data not shown).

When the editing was combined with computational selection of RFP+ cells, however, enriched editing rates of up to 30% were achieved from a single copy delivery CF editing cassette. This enrichment via selection of cells receiving the editing machinery was shown to result in a 2-fold increase in precise, complete intended edits (data not shown). Two or more enrichment/delivery steps can also be used to achieve higher editing rates of CREATE fusion editing in an automated instrument, e.g., use of a module for cell handle enrichment and identification of cells having BFP expression. When the method enriched for cells that have higher CF editing cassette expression levels, the editing rate was even further increased, and thus a growth and/or enrichment module of the instrument may include editing cassette enrichment.

### Example IV: Testing the Effectiveness of RNA Stabilization Moieties in Cells

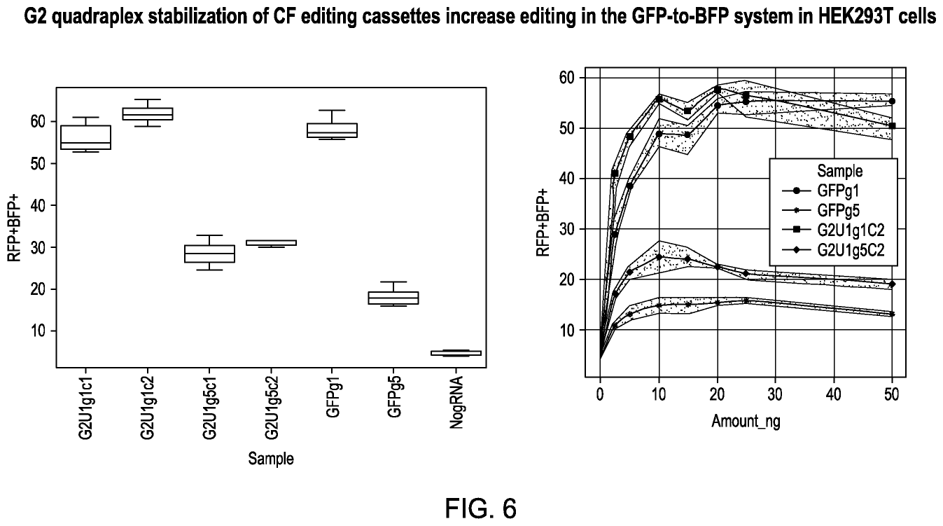

FIG. 6 comprises two graphs demonstrating that CF editing cassettes with a gRNA and with 3′ gRNA stabilization moieties on the repair templates (i.e., “stabilized CF editing cassettes” or “StCFECs”) increase editing in the GFP-to-BFP system. In the graph on the left, “G2U1g1c1” denotes an StCFEC comprising the G2 quadraplex (“G2U1”), a repair template with a 3 bp nick-to-edit distance (“g1”), clone 1; “G2U1g1c2” denotes an StCFEC comprising the G2 quadraplex (“G2U1”), a repair template with a 3 bp nick-to-edit distance (“g1”), clone 2; “G2U1g5cl” denotes an StCFEC comprising the G2 quadraplex (“G2U1”), a repair template with a 20 bp nick-to-edit distance (“g5”), clone 1; “G2U1g5c2” denotes an StCFEC comprising the G2 quadraplex (“G2U1”), repair template with a 20 bp nick-to-edit distance (“g5”), clone 2; GFPg1 denotes a CF editing cassette where the repair template comprises a 3 bp nick-to-edit distance (“g1”) without a stabilization moiety (control); GFPg5 denotes a CF editing cassette where the repair template comprises a 20 bp nick-to-edit distance (“g5”) without a stabilization moiety (control); and NogRNA denotes a control where no CF editing cassette was included in the transduction. The difference between g1 (3 bp) and g5 (20 bp) is the nick-to-edit distance, wherein one would expect stabilization to be more important with a longer nick-to-edit distance. The graph on the right demonstrates that the StCFECs are more efficient at editing than unstabilized CF editing cassettes over a range of different concentrations.

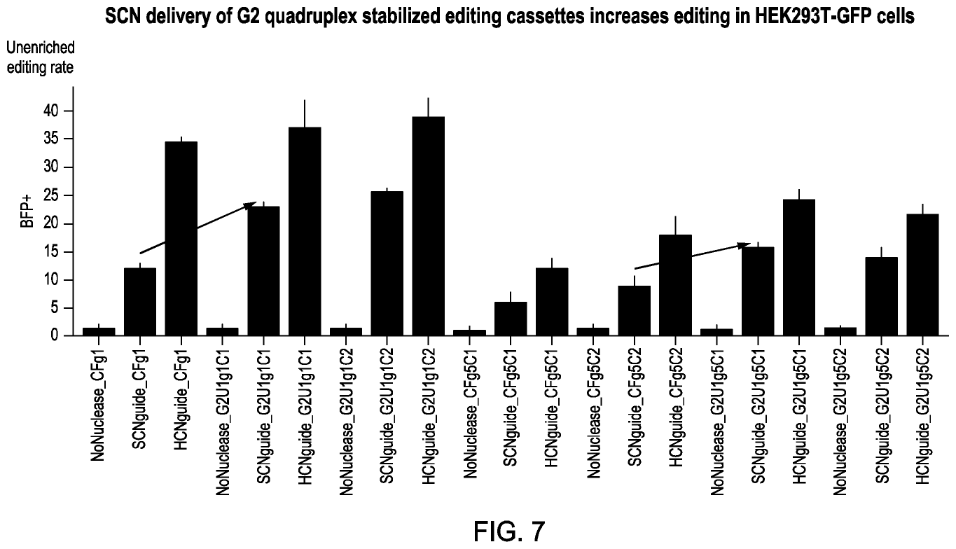

FIG. 7 is a bar graph showing that single copy number lentiviral delivery of StCFECs increases editing over CF editing cassettes (i.e., CF editing cassettes without an RNA stabilization moiety). “SCNguide” denotes a single copy number of the CF editing cassette delivered by lentivirus and integrated into the HEK293T-GFP cell; “HCNguide” denotes a high copy number (2-5 copies) of the CF editing cassette delivered by lentivirus and integrated into the HEK293T-GFP cell; “CFg1” denotes a CF editing cassette where the repair template comprises a 3 bp nick-to-edit distance (“g1”) without an RNA stabilization moiety; “CFg5” denotes a CF editing cassette where the repair template comprises a 3 bp nick-to-edit distance (“g5”) without an RNA stabilization moiety; “NoNuclease” denotes a control using no nuclease; “G2U1” denotes a G2 quadraplex RNA stabilization moiety (see FIG. 1D); and “C1” and “C2” denote clones. Note that there is approximately a 5% increase in editing using HCN G2U1g1 compared to HCN CFg1; a 10% increase in editing using HCN G2U1g5 compared with HCN CFg5; an approximate 12-14% increase in editing with SCN G2U1g1 compared to SCN CFg1; an approximate 10% increase in editing with SCN G2U1g5 compared to SCN CFg5; and the difference in editing between HCN and SON G2U1gRNA (e.g., CF editing cassette stabilized with an RNA moiety) is within 10 to 20%.

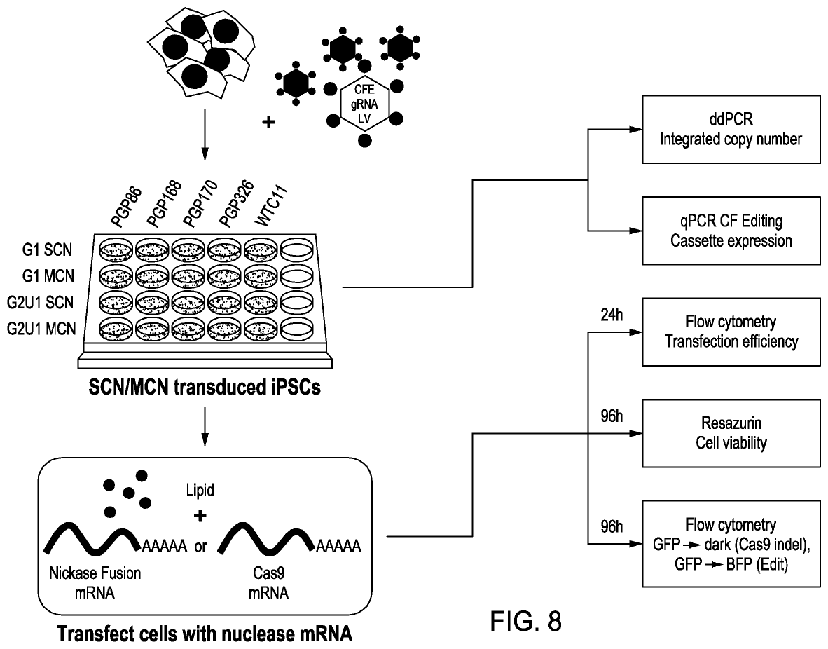

FIG. 8 is a simplified graphic of experimental design for determining cell viability and editing efficiency. For the cell line generation, five iPSC (induced pluripotent stem cell) lines were generated containing a single copy of GFP lentivirus: PGP86; PGP168; PGP170; PGP326; and WTC11. These cell lines were transduced with a GFP CF editing cassette 1 (3 bp nick-to-edit distance) −/+G2 quadraplex lentivirus (G1 vs. G2U1). 1:10,000 and 1:50 lentiviral dilations were used for roughly single-copy (SCN) and multi-copy (MCN) numbers per cell, respectively. The lines were transfected with the nickase-RT fusion mRNA and tested for transfection efficiency and editing. One plate was transfected with Cas9 mRNA to test cutting efficiency and one plate was transfected with lipid only to test transfection viability.

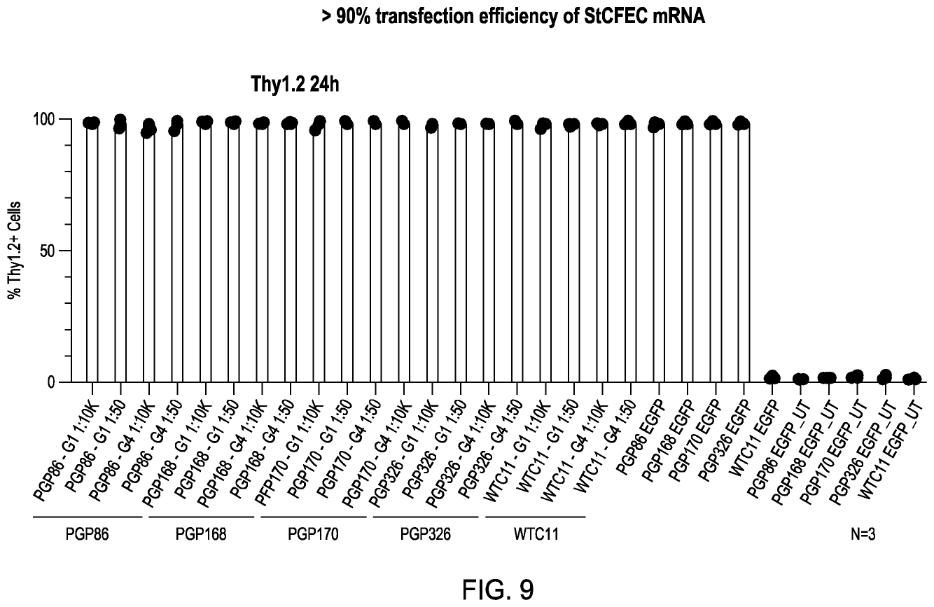

FIG. 9 is a bar graph showing >90% transfection efficiency of StCFECs. Nickase-RT fusion mRNA transfection efficiency was measured by Thy1.2 staining at 24 hours. All cell lines show greater than 90% transfection efficiency. In this figure. “G1” denotes CF editing cassettes without an RNA stabilization moiety; “G4” denotes CF editing cassettes with the G2 quadraplex (e.g., G2U1) RNA stabilization moiety on the 3′ end of the repair template of the CF editing cassette; 1:10K and 1:50 are the lentiviral dilutions for the sample.

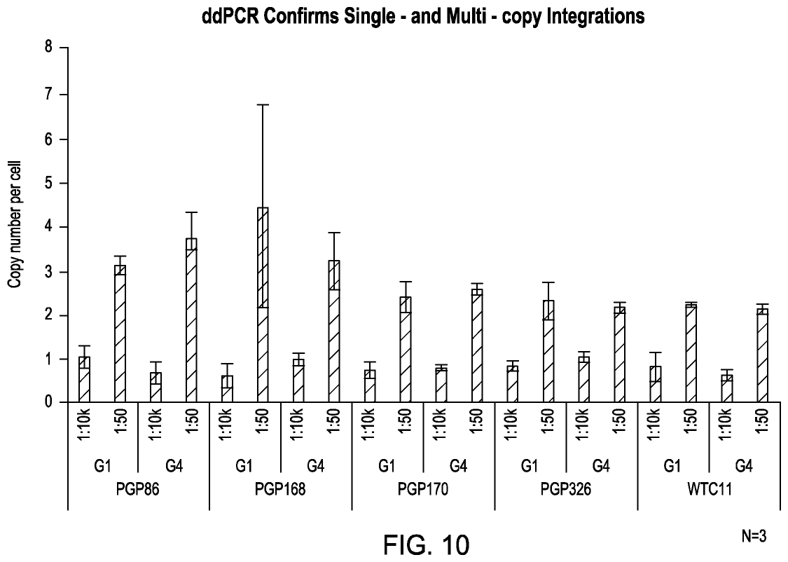

FIG. 10 is a bar graph confirming single- and multiple-copy CF editing cassette integrations by ddPCR. In this figure. “G1” denotes CF editing cassettes without an RNA stabilization moiety; “G4” denotes CF editing cassettes with the G2 quadraplex (e.g., G2U1) RNA stabilization moiety. Copy number was measured by ddPCR using primer-probe sets targeting Chromosome 2 and WPRE (Woodchuck Hepatitis Virus Posttranscriptional Response Element). For editing cassette-integrated cell lines, the copy number was calculated by subtracting the copies detected from the EGFP parental line. EGFP lentivirus was used to create the parental line at roughly 1 copy/cell. Note that 1:10K yields single-copy integration; 1:50 yields an average of 2-4 copies/cell and there was similar transduction efficiency observed across cell lines.

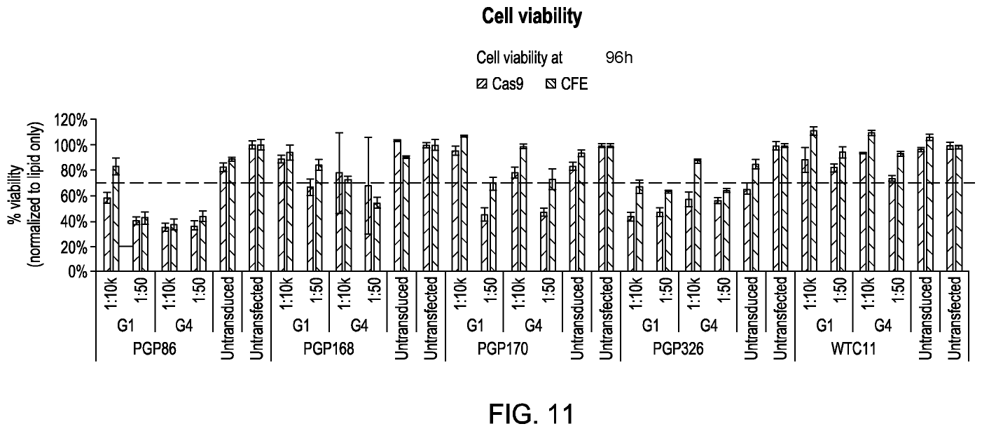

FIG. 11 is a bar graph showing cell viability at 96 hours post-transfection of nuclease mRNA (Cas9 and MAD2007 nickase-RT fusion protein) in different iPSC lines under different lentivirus transfection dilutions. In this figure, “G1” denotes CF editing cassette without an RNA stabilization moiety; “G4” denotes CF editing cassette with the G2 quadraplex (e.g., G2U1) RNA stabilization moiety on the 3′ end of the repair template; “untransduced” denotes a cell line with no editing cassette integrated into the cell line; “untransfected” denotes a cell line that has not been transfected with a nickase-RT fusion or nuclease mRNA. Cell viability was measured by resazyrin at 96 hours and the data was normalized to the respective lipid-only well to account for variability in cell plating. Nickase-RT mRNA transfections show approximately 70% viability, with a generally lower viability in 1:50 dilutions (2-4 copies) than 1:10K dilutions (1 copy). It appears that more editing cassette integration increased the frequency of cut/nick leading to increased cell cycle arrest or apoptosis. The viability of cell lines PCP86 and PGP326 appeared to be more sensitive to mRNA transfections (<70% for most edited samples).

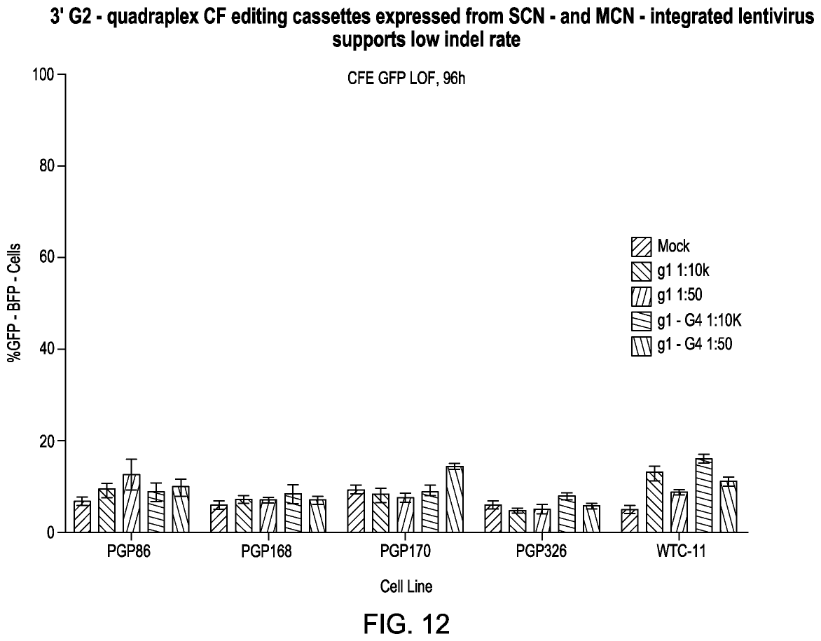

FIG. 12 demonstrates the low indel rates observed in iPSC lines using the MAD2007 nickase-RT fusion protein. “g1-G4” denotes a CF editing cassette with a G2 quadraplex RNA stabilization moiety on the 3′ end of the repair template. There was a 5-10% GFP− background population of cells in each cell line (mock) and a low indel rate was observed in all iPSC lines with CF editing. Indel rates increased in some samples as a function of increased copy number the G2 quadraplex.

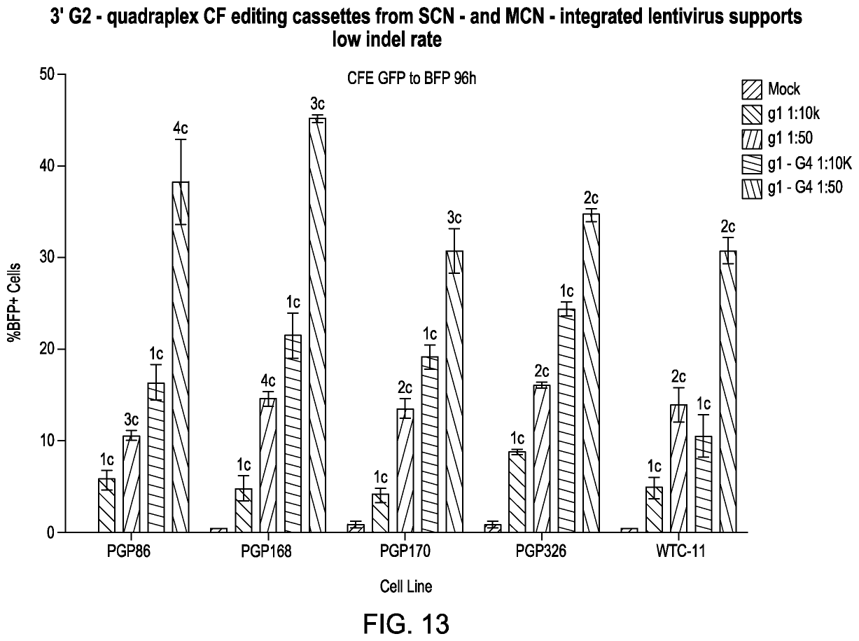

FIG. 13 is a bar graph showing 3′ stabilized CF editing cassettes (StCFECs) of lenti-integrated StCFECs confers robust editing across five iPSC lines. 3′ stabilization of lenti-integrated CF editing cassettes confers robust editing across 5 iPSC lines. Single copy GFP editing cassette integration produces 5-8% editing and increasing copy number yields edit rates of 10-15%. Adding the G2 quadraplex (G2U1) at a single copy increases the edit rate by approximately 3×. Increasing the copy number with G-quadraplex doubles the edit rates compared to single copy by 30% to 43%. Compared to single copy without the G2 quadraplex, there is a 5-8× increase in editing.

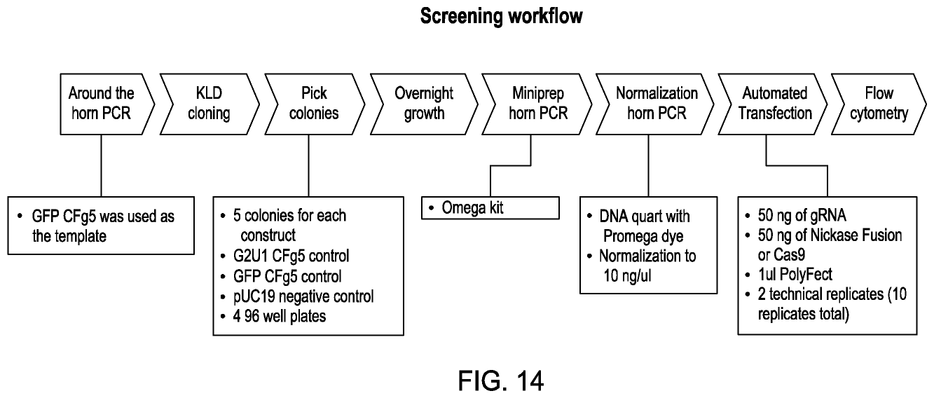

FIG. 14 is a graphic depicting the screening workflow to determine editing efficiency for various putative 3′ stabilization moieties.

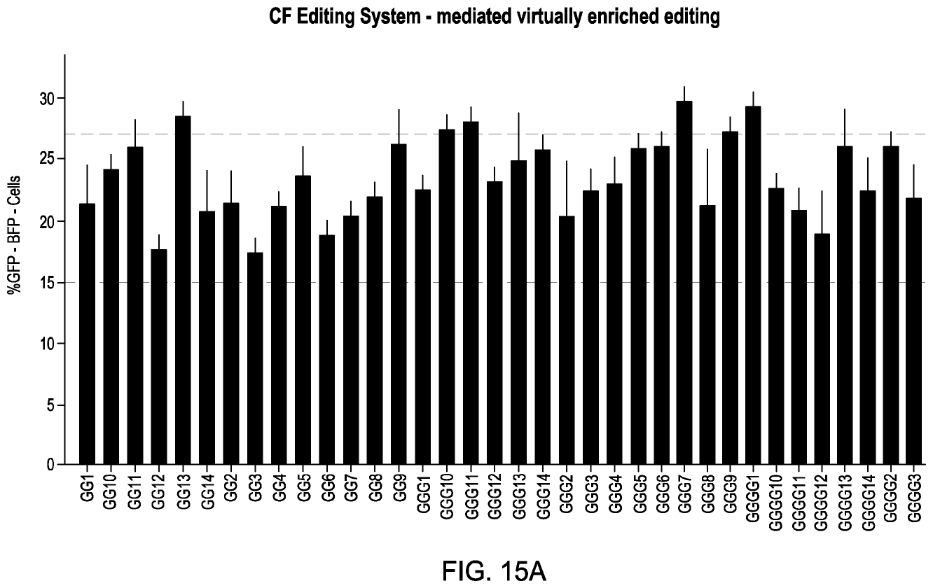

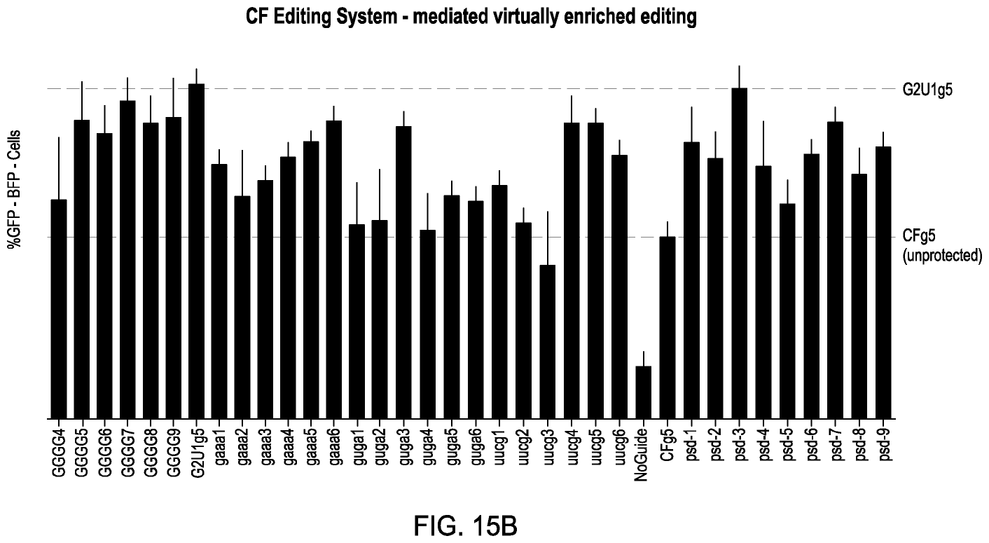

FIGS. 15A and 15B are bar graphs demonstrating the editing rate for the various putative 3′ RNA stabilization moieties listed in Table 1 vis-à-vis the G2U1g5 StCFEC and the CFg5 (unprotected) CF editing cassette.

TABLE 1

| Name of | | | Fold Improvement | |
|---|---|---|---|---|
| Stabilization | Stabilization | Stabilization | over Unprotected | SEQ |
| Moiety | Moiety Class | Moiety Sequence | CFg5 in HEK Cells | ID NO. |
| CFg5 | NA | NA | 1 | |
| G2U1g5 | G-quadraplex | GGTGGTGGTGG | 1.8 | 1 |
| gaaa1 | G-quadraplex | GCCGAAAGGC | 1.4 | 2 |
| gaaa2 | G-quadraplex | GATACCGAAAGGTATC | 1.3 | 3 |
| gaaa3 | G-quadraplex | GATCTGACCGAAAGGTCAGATC | 1.3 | 4 |
| gaaa4 | G-quadraplex | GATCGTCTGACCGAAAGGTCAGACGAT | 1.5 | 5 |
| | | C | | |
| gaaa5 | G-quadraplex | GAGGCTCGTCTGACCGAAAGGTCAGAC | 1.6 | 6 |
| | | GAGCCTC | | |
| gaaa6 | G-quadruplex | GAGGCTTCTAGCGTCTGACCGAAAGGT | 1.7 | 7 |
| | | CAGACGCTAGAAGCCTC | | |
| GG1 | G-quadruplex | GGTGGTGGTGG | 1.4 | 8 |
| GG10 | G-quadraplex | GGAGGAGGAGGAGGAGGAGG | 1.6 | 9 |
| GG11 | G-quadraplex | GGAGGTGGAGGTGGAGGTGG | 1.7 | 10 |
| GG12 | G-quadraplex | GGCCTGTGGCCTGTGGCCTGTGG | 1.2 | 11 |
| GG13 | G-quadraplex | GGCCTGTTGGCCTGTTGGCCTGTTGG | 1.9 | 12 |
| GG14 | G-quadraplex | GGTAGCATTGGTAGCATTGGTAGCATT | 1.4 | 13 |
| | | GG | | |
| GG2 | G-quadraplex | GGAGGAGGAGG | 1.4 | 14 |
| GG3 | G-quadruplex | GGCGGCGGCGG | 1.2 | 15 |
| GG4 | G-quadraplex | GGAAGGAAGGAAGG | 1.4 | 16 |
| GG5 | G-quadraplex | GGCTGGCTGGCTGG | 1.6 | 17 |
| GG6 | G-quadraplex | GGCAGGCAGGCAGG | 1.3 | 18 |
| GG7 | G-quadraplex | GGAGAGGAGAGGAGAGG | 1.4 | 19 |
| GG8 | G-quadraplex | GGAAAGGAAAGGAAAGG | 1.5 | 20 |
| GG9 | G-quadraplex | GGTCAGGTCAGGTCAGG | 1.8 | 21 |
| GGG1 | G-quadraplex | GGGTGGGTGGGTGGG | 1.5 | 22 |
| GGG10 | G-quadraplex | GGGAGGAGGGAGGAGGGAGGAGGG | 1.8 | 23 |
| GGG11 | G-quadraplex | GGGAGGTGGGAGGTGGGAGGTGGG | 1.9 | 24 |
| GGG12 | G-quadraplex | GGGCCTGTGGGCCTGTGGGCCTGTGGG | 1.6 | 25 |
| GGG13 | G-quadraplex | GGGCCTGTTGGGCCTGTTGGGCCTGTT | 1.7 | 26 |
| | | GGG | | |
| GGG14 | G-quadraplex | GGGTAGCATTGGGTAGCATTGGGTAGC | 1.7 | 27 |
| | | ATTGGG | | |
| GGG2 | G-quadraplex | GGGGGGAGGGAGGG | 1.4 | 28 |
| GGG3 | G-quadraplex | GGGCGGGCGGGCGGG | 1.5 | 29 |
| GGG4 | G-quadraplex | GGGAAGGGAAGGGAAGGG | 1.5 | 30 |
| GGG5 | G-quadraplex | GGGCTGGGCTGGGCTGGG | 1.7 | 31 |
| GGG6 | G-quadraplex | GGGCAGGGCAGGGCAGGG | 1.7 | 32 |
| GGG7 | G-quadraplex | GGGAGAGGGAGAGGGAGAGGG | 2 | 33 |
| GGG8 | G-quadraplex | GGGAAAGGGAAAGGGAAAGGG | 1.5 | 34 |
| GGG9 | G-quadraplex | GGGTCAGGGTCAGGGTCAGGG | 1.8 | 35 |
| GGGG1 | G-quadraplex | GGGGTGGGGTGGGGGGGG | 2 | 36 |
| GGGG10 | G-quadruplex | GGGGAGGAGGGGAGGAGGGGAGGAG | 1.6 | 37 |
| | | GGG | | |
| GGGG11 | G-quadraplex | GGGGAGGTGGGGAGGTGGGGAGGTGG | 1.4 | 38 |
| | | GG | | |
| GGGG12 | G-quadraplex | GGGGCCTGTGGGGCCTGTGGGGCCTGT | 1.3 | 39 |
| | | GGGG | | |
| GGGG13 | G-quadraplex | GGGGCCTGTTGGGGCCTGTTGGGGCCT | 1.8 | 40 |
| | | GTTGGGG | | |
| GGGG14 | G-quadraplex | GGGGTAGCATTGGGGTAGCATTGGGGT | 1.5 | 41 |
| | | AGCATTGGGG | | |
| GGGG2 | G-quadraplex | GGGGAGGGGAGGGGGGGG | 1.8 | 42 |
| GGGG3 | G-quadraplex | GGGGGGGGGGGGGCGGGG | 1.5 | 43 |
| GGGG4 | G-quadruplex | GGGGAAGGGGAAGGGGAAGGGG | 1.2 | 44 |
| GGGG5 | G-quadraplex | GGGGCTGGGGCTGGGGCTGGGG | 1.7 | 45 |
| GGGG6 | G-quadraplex | GGGGCAGGGGCAGGGGCAGGGG | 1.6 | 46 |
| GGGG7 | G-quadraplex | GGGGAGAGGGGAGAGGGGAGAGGGG | 1.8 | 47 |
| GGGG8 | G-quadraplex | GGGGAAAGGGGAAAGGGGAAAGGGG | 1.7 | 48 |
| GGGG9 | G-quadraplex | GGGGTCAGGGGTCAGGGGTCAGGGG | 1.7 | 49 |
| guga1 | Hairpin | GCCGTGAGGC | 1.1 | 50 |
| guga2 | Hairpin | GATACCGTGAGGTATC | 1.1 | 51 |
| guga3 | Hairpin | GATCTGACCGTGAGGTCAGATC | 1.6 | 52 |
| guga4 | Haimpin | GATCGTCTGACCGTGAGGTCAGACGAT | 1 | 53 |
| | | C | | |
| guga5 | Hairpin | GAGGCTCGTCTGACCGTGAGGTCAGAC | 1.2 | 54 |
| | | GAGCCTC | | |
| guga6 | Hairpin | GAGGCTTCTAGCGTCTGACCGTGAGGT | 1.2 | 55 |
| | | CAGACGCTAGAAGCCTC | | |
| psd-1 | Pseudoknot | GCGACTTCGGTCGCCGAA | 1.5 | 56 |
| psd-2 | Pseudoknot | GCGACTTCGCATGTCGCATGCGAA | 1.4 | 57 |
| psd-3 | Psendoknot | GCGACTTCGCATAGACGTCGCGTCTAT | 1.8 | 58 |
| | | GCGAA | | |
| psd-4 | Pseudoknot | GCGACTAGTTCGCTAGTCGCCGAA | 1.4 | 59 |
| psd-5 | Pseudoknot | GCGACTAGTTCGCATCTAGTCGCATGC | 1.2 | 60 |
| | | GAA | | |
| psd-6 | Pseudoknot | GCGACTAGTTCGCATAGACCTAGTCGC | 1.5 | 61 |
| | | GTCTATGCGAA | | |
| psd-7 | Pseudoknot | GAGCTAGCATCATTCGTGATGCTAGCT | 1.6 | 62 |
| | | CCGAA | | |
| psd-8 | Pseudoknot | GAGCTAGCATCATTCGCATTGATGCTA | 1.4 | 63 |
| | | GCTCATGCGAA | | |
| psd-9 | Pseudoknot | GAGCTAGCATCATTCGCATAGACTGAT | 1.5 | 64 |
| | | GCTAGCTCGTCTATGCGAA | | |
| uucg1 | Hairpin | GCCTTCGGGC | 1.3 | 65 |
| uucg2 | Hairpin | GATACCTTCGGGTATC | 1.1 | 66 |
| uucg3 | Hairpin | GATCTGACCTTCGGGTCAGATC | 0.9 | 67 |
| uucg4 | Hairpin | GATCGTCTGACCTTCGGGTCAGACGAT | 1.6 | 68 |
| | | C | | |
| uucg5 | Hairpin | GAGGCTCGTCTGACCTTCGGGTCAGAC | 1.6 | 69 |
| | | GAGCCTC | | |
| uucg6 | Hairpin | GAGGCTTCTAGCGTCTGACCTTCGGGT | 1.5 | 70 |
| | | CAGACGCTAGAAGCCTC | | |
| TABV | exoribonuclease | GGCAAGGTACGGATTAGCCGTAGGGG | 1 | 71 |
| | resistant | CTTGAGAACCCCCCCTCCCCACTC | | |
| | RNA | | | |
| TBBV | exoribonuclease | CACAGATCATGGAATGATGCGGCAGC | 1.2 | 72 |
| | resistant | GCGCGAGAGCGACGGGGAAGTGGTCG | | |
| | RNA | CACCCGACGCACCATCCATGAAGCAAT | | |
| | | ACTTCGTGAGACCC | | |
| ZIKV | exoribonuclease | GGGTCAGGCCGGCGAAAGTCGCCACA | 1.6 | 73 |
| | resistant | GTTTGGGGAAAGCTGTGCAGCCTGTAA | | |
| | RNA | CCCC | | |

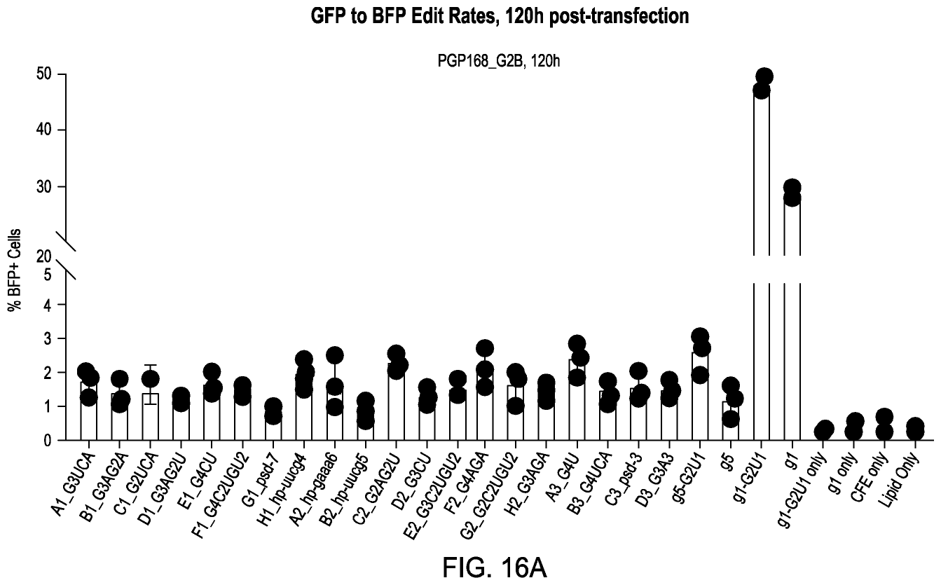

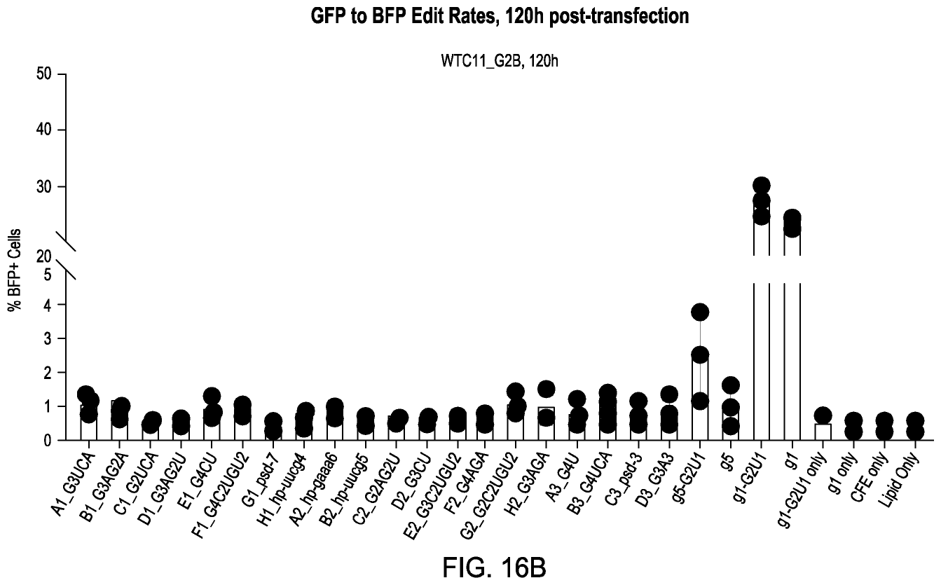

FIG. 16A is a bar graph of GFP to BFP edit rates 120 hours post-transfection in PGP168 iPSCs. FIG. 16B is a bar graph of GFP to BFP edit rates 120 hours post-transfection in WTC11 iPSCs. Note that the G2U1 G-quadraplex improves edit rates for both g1 (3 bp nick-to-edit) and g5 (20 bp nick-to-edit); edit rates from approximately 0.5% to 2.5% are observed with CF editing cassette g5 (no RNA stabilization moiety); some RNA stability elements approach edit rates observed with G2U1 although none outperform G2U1; there are lower overall edit rates observed in WTC11 vs. PGP168, which is consistent with the lower transfection efficiency; and the same trend for g1, g5+/G2U1 is observed across cell lines.

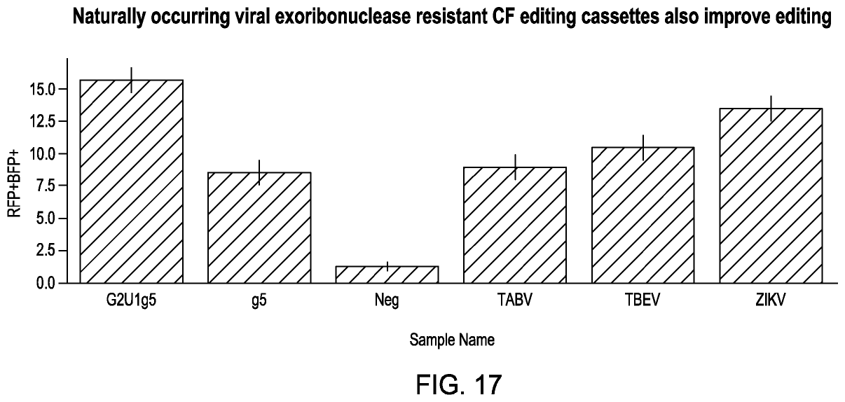

FIG. 17 shows the improvement in editing rates for viral exoribonuclease resistant RNAs used as 3′ stabilization moieties. Exoribonuclease resistant RNAs (xrRNAs) are a class of RNAs found in flaviviruses at the 3′ UTS region of the viral genome, with a role to provide exoribonuclease protection. TABV, TBEV and ZIKV xrRNAs were appended to the 3′ end of a GFP CF editing cassette and compared to a G2U1-protected CF editing cassette with a 20 bp nick-to-edit distance (G2U1g5) and compared to a CF editing cassette comprising a 20 bp nick-to-edit distance (g5) and no protection on the 3′ end.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, ¶6.

1.-19. (canceled)

20. A method for editing a cell, the method comprising:
(a) introducing a CREATE fusion editing cassette for performing nucleic acid-guided nickase/reverse transcriptase fusion editing to the cell, wherein the CREATE fusion editing cassette comprises, from 3′ to 5′:
(i) an RNA repair template, wherein the RNA repair template comprises:
(A) an RNA stabilization moiety;
(B) a linker region;
(C) a primer binding region capable of binding to a nicked target DNA;
(D) a nick-to-edit-region; and
(E) a region of post-edit homology; and
(ii) a guide RNA (gRNA), wherein the guide RNA comprises:
(A) a guide sequence; and
(B) a scaffold region; and
(b) providing the cell with conditions that allow:
(i) the scaffold region of the gRNA to interact or complex with a nucleic acid-guided nickase/reverse transcriptase fusion protein to form a ribonucleoprotein complex; and
(ii) the guide sequence of the gRNA to hybridize to a genomic target locus, and wherein the ribonucleoprotein complex edits the genomic target locus within the cell.

21. The method of claim 20, wherein the RNA stabilization moiety is selected from the group consisting of a G quadraplex, an RNA hairpin, an RNA pseudoknot, and an exoribonuclease resistant RNA.

22. The method of claim 21, wherein the G quadraplex comprises a nucleic acid sequence selected from the group consisting of SEQ ID NOs: 1 to 49.

23. The method of claim 21, wherein the RNA hairpin comprises a nucleic acid sequence selected from the group consisting of SEQ ID NOs: 50 to 55 and 65 to 70.

24. The method of claim 21, wherein the RNA pseudoknot comprises a nucleic acid sequence selected from the group consisting of SEQ ID NOs: 56 to 64.

25. The method of claim 21, wherein the exoribonuclease resistant RNA comprises a nucleic acid sequence selected from the group consisting of SEQ ID NOs: 71 to 73.

26. The method of claim 20, wherein the linker region comprises between 0 nucleotides and 20 nucleotides.

27. The method of claim 20, wherein the primer binding region comprises between 0 nucleotides and 20 nucleotides.

28. The method of claim 20, wherein the nick-to-edit region comprises between 0 nucleotides and 20 nucleotides.

29. The method of claim 20, wherein the region of post-edit homology comprises between 3 nucleotides and 20 nucleotides.

30. The method of claim 20, wherein the CREATE fusion editing cassette further comprises a barcode.

31. The method of claim 20, wherein the CREATE fusion editing cassette further comprises a nucleic acid sequence encoding a selectable marker.

32. The method of claim 20, wherein the CREATE fusion editing cassette is under the control of an inducible promoter.

33. The method of claim 20, wherein the CREATE fusion editing cassette is provided to the cell as a messenger RNA molecule.

34. The method of claim 20, wherein the cell is a eukaryotic cell.

35. The method of claim 20, wherein the cell is a prokaryotic cell.

36. The method of claim 35, wherein the prokaryotic cell is an Escherichia coli cell.

37. The method of claim 20, wherein the nucleic acid-guided nickase is a MAD2007 nickase.

38. The method of claim 20, wherein the introducing comprises a delivery system selected from the group consisting of a yeast system, a lipofection system, a microinjection system, a biolistic system, a virosome, a liposome, an immunoliposome, a polycation, a lipid:nucleic acid conjugate, a virion, an artificial virion, a viral vector, electroporation, a cell permeable peptide, a nanoparticle, a nanowire, and an exosome.

39. The method of claim 20, wherein the method takes place within an automated cell editing instrument.
